(19) United States
(12) Patent Application Publication
(10) Pub. No.: US 2026/0014274 A1
(43) Pub. Date: Jan. 15, 2026

(54) REVERSIBLE, COVALENT POLYNUCLEOTIDE CONDENSATION APPROACH FOR ENHANCED GENE DELIVERY

(71) Applicant: BOARD OF REGENTS, THE UNIVERSITY OF TEXAS SYSTEM, Austin, TX (US)

(72) Inventors: Huiliang WANG, Austin, TX (US); Wenliang WANG, Austin, TX (US); Aaron TASSET, Austin, TX (US)

(21) Appl. No.: 18/881,470

(22) PCT Filed: Jul. 6, 2023
(86) PCT No.: PCT/US2023/027025

§ 371 Date: Jan. 6, 2025

Related U.S. Application Data
(60) Provisional application No. 63/358,681, filed on Jul. 6, 2022.

(51) Int. Cl. A61K 48/00

(52) U.S. Cl. CPC A61K 48/0041; A61K 48/0058; A61K 48/0091

(57) ABSTRACT

The present invention relates to compositions and methods for delivering nucleic acids into cells.

### I. CROSS-REFERENCE TO RELATED APPLICATIONS

This application claims the benefit of U.S. Provisional Application No. 63/358,681, filed Jul. 6, 2022, which is expressly incorporated herein by reference in its entirety.

### II. BACKGROUND

Gene therapy is a tool to explore pathogenesis and a powerful weapon to treat diseases at the genomic level. Over the past decades it has been utilized in chimeric antigen receptor (CAR) based cell therapeutics, optogenetics to study the neural circuit, and gene treatments of inherited diseases and neurodegenerative diseases. Viral vectors, such as adeno-associated vectors (AAV), have become a widely exploited vehicle to express exogenous DNA into targeting cells due to their high transfection efficiency. For example, AAV vectors used to treat congenital blindness have been approved via Food and Drug Administration (FDA) since 2017. However, several setbacks of viral vectors severely limited the gene therapy in clinical trials; including immunogenicity, carcinogenesis, limited DNA packaging capacity (DNA<5 Kbp) and high production cost. Non-viral vectors have great potential to address many of the limitations of viral vectors, particularly with respect to cost and safety. Most cationic polymers, such as poly(β-amino ester) s polymers, polypeptides, dendrimer polymers, polyethylenimine derivatives and gold nanoparticles have been developed to deliver genes, including siRNA, mRNA, and DNA. Recently, lipid nanoparticles-based mRNA vaccines have been successfully developed against the COVID.

While great developments have been achieved, the transfection efficiency of non-viral vectors in DNA is not sufficient for clinical needs. The cationic non-viral vector usually exhibited dose-dependent performance, where the DNA complexes were easily dissociated and lost transfection ability upon dilution, but the high dose of cationic vectors were often associated with strong cytotoxicity. In addition, rare gene transcriptions are initiated when the DNA size is larger than 7 Kbp due to the loose condensation and inefficient nucleus transport of cationic non-viral vectors. Fundamentally, efficient gene transfer requires not simply the entry of DNA from the extracellular surface of the cell into the cytoplasm, but also delivery across the nuclear envelope and into the nucleus before any transcription can initiate. In current non-viral vector strategy, native DNA with larger size is mainly condensed and packaged into the cationic nanoparticles via electrostatic interaction in high vectors concentration, and then released in the cytoplasm after escaping from endosomes via proton sponge effect. However, while viral vectors could enable direct nuclear entry of DNAs, most of the released DNA from non-viral vectors could not simply diffuse through the viscous cytoplasm and enter the nucleus via ˜60 nm diameter nuclear envelope channels to initiate transcription before degradation. To address these challenges, the linear mini DNA strategy has been developed to decrease the DNA size and improve the nucleus targeting efficiency, while the mini DNAs are usually less stable than DNA plasmids and the high cost and complicated process of the mini DNAs purification dramatically hamper their applications. Additionally, nucleus targeting proteins have also been conjugated to the genes to achieve nucleus delivery, but this strategy also puts heavy demands on the gene modification process and causes high cost. What are needed are new method for gene modification.

### III. SUMMARY

Disclosed herein relate to nanocomposites, methods of producing said nanocomposites, and uses of the nanocomposites for modulation of nucleic acid levels in cells. The nanocomposites are produced via a two-stage condensation approach to achieve efficient gene delivery. The native nucleic acid reacts with crosslinker, and are condensed into bundled nucleic acid (b-DNA or b-RNA) with smaller size via reversible covalent self-assembly. Then, these bundled nucleic acids can be compressed again by any cationic delivery systems via electrostatic interaction to form complexes. After the condensation, the nucleic acid can be more easily diffuse into cytoplasm and enter the nucleus, thus increasing the gene transfection efficiency.

Accordingly, in some aspects, disclosed herein is a nanocomposite comprising
a) a cationic vector; and
b) a chemically modified nucleic acid that comprises at least one nucleobase that is linked to a chemical crosslinker via a reversible covalent bond.

In some embodiments, the reversible covalent bond is a carbamate bond. The reversible covalent bond can link the chemical crosslinker to an amine of the at least one nucleobase. In some embodiments, the chemical crosslinker is a p-nitrophenylcarbonate crosslinker (e.g., disulfides (ethane-2,1-diyl)bis(4-nitrophenyl) bis(carbonate) (NPC)). In some embodiments, the chemical crosslinker is PEGylated (e.g., a PEGylated NPC crosslinker). In some embodiments, the PEGylated NPC crosslinker has a molecular weight of about 1000 g/mol, 2000 g/mol, 3000 g/mol, 3400 g/mol, or 5000 g/mol. In some embodiments, the PEGylated NPC crosslinker is a 4-arm PEGylated NPC crosslinker, an 8-arm PEGylated NPC crosslinker, or a 16-arm PEGylated NPC crosslinker.

In some embodiments, the chemically modified nucleic acid forms a bundle that is less than about 150 nm in size. In some embodiments, the cationic vector is a cationic polymer or a cationic liposome.

In some embodiments, the nucleic acid is a DNA or an RNA. In some embodiments, the nucleic acid is at least 5000 bp in length.

Also disclosed herein is a pharmaceutical composition comprising the nanocomposite of any preceding aspect.

Also disclosed herein is a kit for introducing a nucleic acid into a cell, said kit comprising the nanocomposite of any preceding aspect.

In some aspects, disclosed herein is a method of producing a nanocomposite for delivering a nucleic acid, said method comprising
a) providing a chemical crosslinker;
b) contacting the nucleic acid with the chemical crosslinker thereby creating a chemically modified nucleic acid that comprises at least one nucleobase that is linked to a chemical crosslinker via a reversible covalent bond; and
c) contacting the chemically modified nucleic acid with a cationic vector thereby packaging the chemically modified nucleic acid into the cationic vector to produce the nanocomposite.

In some embodiments, the reversible covalent bond is a carbamate bond, an imides bond, or an amides bond. The reversible covalent bond can link the chemical crosslinker to an amine of the at least one nucleobase. In some embodiments, the chemical crosslinker is a p-nitrophenylcarbonate crosslinker (e.g., disulfides (ethane-2,1-diyl)bis(4-nitrophenyl) bis(carbonate) (NPC)). In some embodiments, the chemical crosslinker is PEGylated (e.g., a PEGylated NPC crosslinker). In some embodiments, the PEGylated NPC crosslinker has a molecular weight of about 1000 g/mol, 2000 g/mol, 3000 g/mol, 3400 g/mol, or 5000 g/mol. In some embodiments, the PEGylated NPC crosslinker is a 4-arm PEGylated NPC crosslinker, an 8-arm PEGylated NPC crosslinker, or a 16-arm PEGylated NPC crosslinker.

In some embodiments, the chemically modified nucleic acid forms a bundle that is less than about 150 nm in size. In some embodiments, the cationic vector is a cationic polymer or a cationic liposome.

In some embodiments, the cationic polymer and the chemically modified nucleic acid is in contact at a nitrogen/phosphorus (N/P) ratio of about 5:1, 10:1, or 15:1.

In some embodiments, the cationic liposome and the chemically modified nucleic acid is in contact at a weight ratio of about 1:1, 1:2, or 1:3.

In some embodiments, the nucleic acid and the chemical crosslinker is in contact at a ratio in weight of about 1:4.

In some aspects, disclosed herein is a method of introducing a nucleic acid into a cell, said method comprising
a) providing the nanocomposite of or the pharmaceutical composition of any preceding aspect, wherein the nanocomposite or the pharmaceutical composition comprises the nucleic acid; and
b) contacting the nanocomposite with the cell thereby introducing nucleic acid into the cell.

In some aspects, disclosed herein is a method of modulating the level of a nucleic acid in a cell in a subject in need, said method comprising
a) providing the nanocomposite of or the pharmaceutical composition of any preceding aspect, wherein the nanocomposite or the pharmaceutical composition comprises the nucleic acid; and
b) administering a therapeutically effective amount of the nanocomposite to the subject in need.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1(a-d). Schematic shows reversible covalent assembly strategy (RECAST) condenses the DNA through covalent linkers to achieve direct nucleus delivery and traceless release. (FIG. 1a) Free amino groups are exposed in the surface of DNA, which could be reacted with disulfides (ethane-2,1-diyl)bis(4-nitrophenyl) bis(carbonate) (NPC) functional crosslinkers (C1, C2 and C3, with the chemical details shown in FIG. 9) to condense DNA below to the nucleopores central channel (<60 nm) to form smaller b-DNA. (FIG. 1b) The b-DNA is compressed into cationic PEI via electrostatic interaction to form complexes of 100 nm. (FIG. 1c) After entering the cytoplasm, b-DNA cargoes are released and diffused into the nucleus via nucleopores. (FIG. 1d) Finally, reductive cleavage of the disulfide will result in carbamate cleavage to tracelessly release the DNA plasmid for gene transcription.

FIG. 2(a-e). The hydration radius of DNA plays a crucial role in gene transfection. (FIG. 2a) the hydration size of DNA plasmids in PBS detected via Dynamic Light Scattering (DLS) (n=3 per group, one-way ANOVA); (FIG. 2b) the hydration size of PEI/DNA complexes at different N/P (n=3 per group, one-way ANOVA); (FIG. 2c) images of GFP expression following transfection with PEI/DNA complexes at N/P of 10: (i) 4.5 Kbp DNA, (ii) 6.2 Kbp DNA and (iii) 11.6 Kbp DNA, Scale bar: 100 μm; (FIG. 2d) the fluorescence intensity quantification of PEI/DNA complexes transfection images at N/P of 10 (n>5 per group, one-way ANOVA); (FIG. 2e) the flow cytometry quantification analysis of PEI/DNA complexes at N/P of 10 (n>4 per group, one-way ANOVA). All plots show mean±SEM unless otherwise mentioned. *P<0.05, **P<0.01, ***P<0.001, ****P<0.0001; ns, not significant.

FIG. 3(a-f). DNA condensation via RECAST. (FIG. 3a) The Dynamic Light Scattering (DLS) tests of original DNA and b-DNA; (FIG. 3b) The measurements of DNA crosslinking density and DNA hydration size changes with the reaction time, where crosslinker PEG₂₀₀₀ is used; (FIG. 3c) The measurements of DNA crosslinking density and DNA surface potential changes with the reaction time, where crosslinker PEG₂₀₀₀ is used; (FIG. 3d) The crosslinking density of DNA after reacting with various linkers for 24 hours; (FIG. 3e) The final DNA hydration size after reacting with different linkers for 24 hours (n=3 per group, one-way ANOVA); (FIG. 3f) The final DNA surface potential after reacting with various linkers for 24 hours (n=3 per group, one-way ANOVA). All plots show mean±SEM unless otherwise mentioned. *P<0.05, **P<0.01, ***P<0.001, ****P<0.0001; ns, not significant.

FIG. 4(a-h). Enhanced green fluorescence protein (GFP) expression of 6.2 kbp and 11.2 kbp b-DNA through RECAST in HEK cells. (FIG. 4a) GFP expression images of HEK cells using PEI/DNA (6.2 Kbp) complexes at N/P of 5 for (i) original DNA, and condensed DNA via (ii) NPC linker, (iii) 4-arm-PEGylated NPC linker, (iv) PEG₁₀₀₀ NPC linker, (v) PEG₂₀₀₀ NPC linker, (vi) PEG₃₄₀₀ NPC linker, (vii) PEG₅₀₀₀ NPC linker, scale bar: 100 μm; and (FIG. 4b) the corresponding fluorescence intensity quantification analysis of (FIG. 4a) (n=4 per group, one-way ANOVA); (FIG. 4c) the flow cytometry quantification analysis of various PEI/b-DNA complexes at N/P of 5 (n>4 per group, one-way ANOVA); (FIG. 4d) the cell viability flow cytometry quantification analysis of various PEI/b-DNA complexes at different N/P ratios (n>3 per group, one-way ANOVA); (FIG. 4e) GFP expression images of HEK cells by various PEI/b-DNA (11.2 kbp) complexes at N/P of 5, (i) original DNA, and condensed DNA via (ii) NPC linker, (iii) 4-arm-PEGylated NPC linker, (iv) PEG₁₀₀₀ NPC linker, (v) PEG₂₀₀₀ NPC linker, (vi) PEG₃₄₀₀ NPC linker, (vii) PEG₅₀₀₀ NPC linker, scale bar: 100 μm; (FIG. 4f) the corresponding fluorescence intensity quantification analysis of (FIG. 4a) (n=3 per group, one-way ANOVA); (FIG. 4g) the flow cytometry quantification analysis of PEI/b-DNA (11.6 kbp) complexes with different chemical linkers at N/P of 5 (n=3 per group, one-way ANOVA); (FIG. 4h) the flow cytometry quantification analysis of various PEI/b-DNA (11.2 kbp) complexes at different N/P ratios (n=3 per group, one-way ANOVA). All plots show mean±SEM unless otherwise mentioned. *P<0.05, **P<0.01, ***P<0.001, ****P<0.0001; ns, not significant.

FIG. 5(a-b). Enhanced green fluorescence protein (GFP) expression of 6.2 kbp b-DNA through RECAST in primary cultured neurons. (FIG. 5a) GFP expression images of primary neurons using polymer/DNA (6.2 kbp) complexes, where the neuron nucleuses were stained with NeuN (red) and the total cell population nucleuses with DAPI (blue). Scale bar: 100 μm; (FIG. 5b) the corresponding statistics quantification analysis of (FIG. 5a) (n>2 per group, one-way ANOVA).

FIG. 6(a-d). Enhanced nucleus transportation and traceless release of b-DNA in an intracellular high-GSH environment. (FIG. 6a) Confocal Laser Scanning Microscopy (CLSM) images of HEK293 cells incubated with Cy5-labeled b-DNA and original DNA at PEI N/P of 10. Cy5-DNA was shown in red, and the nucleus was in blue, scale bar: 20 μm; (FIG. 6b) the quantitative analysis of Cy5 dots overlapped with nucleus (n>4 per group, one-way ANOVA); (FIG. 6c) the size changes of b-DNA after incubation for different hours in 10 mM GSH solution; (FIG. 6d) The gene sequence tests of (i) original DNA, and (ii) released DNA after incubation for 6 h in 10 mM GSH solution. All plots show mean±SEM unless otherwise mentioned. *P<0.05, **P<0.01, ***P<0.001, ****P<0.0001; ns, not significant.

FIG. 7(a-d). Enhanced in vivo gene delivery efficiency in targeted organs after intravenous administration through RECAST. (FIG. 7a) Bioluminescent images of mice taken with IVIS after 6 h post-injection. (FIG. 7b) Statistical analysis of bioluminescent radiance after 6 h post-injection (n=5, one-way ANOVA, Tukey post-hoc testing). (FIG. 7c) Quantification of luciferase expression in the body over time post-treatment (n=5). (FIG. 7d) Bioluminescent images of extracted organs (heart, liver, spleen, lung, kidney, stomach and intestine) 24 h post-treatment, all plots show mean±SEM. *P<0.05.

FIG. 8. Schematic showing the steps.

FIG. 9. The chemical structure of C1, C2 and C3 crosslinkers.

FIG. 10(a-b). (FIG. 10a) The DLS tests of three plasmids in PBS; (FIG. 10b) the zeta potential of PEI/DNA complexes at different N/P ratios (n=3, per group).

FIG. 11(a-f). The GFP expression of PEI/DNA at N/P of 5 and of 15. (FIG. 11a) The gene transfection efficiency of PEI/DNA complexes at N/P of 5, (i) 4.5 Kbp, (ii) 6.2 Kbp, (iii) 11.6 Kbp; (FIG. 11b) the fluorescence intensity quantification of PEI/DNA complexes transfection images at N/P of 5 (n>5 per group, one-way ANOVA); (FIG. 11c) the flow cytometry quantification analysis of PEI/DNA complexes at N/P of 5 (n=4 per group, one-way ANOVA). (FIG. 11d) The gene transfection efficiency of PEI/DNA complexes at N/P of 15, (i) 4.5 Kbp, (ii) 6.2 Kbp, (iii) 11.6 Kbp. scale bar: 100 μm; (FIG. 11e) the fluorescence intensity quantification of PEI/DNA complexes transfection images at N/P of 15 (n>5 per group, one-way ANOVA); (FIG. 11f) the flow cytometry quantification analysis of PEI/DNA complexes at N/P of 15 (n=4 per group, one-way ANOVA). All plots show mean±SEM unless otherwise mentioned. *P<0.05, **P<0.01, ***P<0.001, ****P<0.0001; ns, not significant.

FIG. 12(a-c). The GFP expression of Lipofectamine 2000/DNA at ratio of 1:1, 2:1 and 3:1. (FIG. 12a) The gene transfection efficiency of Lipofectamine 2000/DNA (4.5 Kbp) complexes at (i) ratio 1:1, (ii) ratio 2:1, and (iii) ratio 3:1, w/w; and (iv) the fluorescence intensity quantification (n>4 per group, one-way ANOVA) (v) the flow cytometry quantification analysis at ratio 1:1 (n>3 per group, one-way ANOVA); (FIG. 12b) The gene transfection efficiency of PEI/DNA complexes (6.2 Kbp) at (i) ratio 1:1, (ii) ratio 2:1, and (iii) ratio 3:1, w/w; and (iv) the fluorescence intensity quantification (n>4 per group, one-way ANOVA) (v) the flow cytometry quantification analysis at ratio 2:1 (n>3 per group, one-way ANOVA); (FIG. 12c) The gene transfection efficiency of PEI/DNA complexes (11.6 Kbp) at (i) ratio 1:1, (ii) ratio 2:1, and (iii) ratio 3:1, w/w, scale bar: 100 μm; and (iv) the fluorescence intensity quantification (n>4 per group, one-way ANOVA) (v) the flow cytometry quantification analysis at ratio 3:1 (n>3 per group, one-way ANOVA); All plots show mean±SEM unless otherwise mentioned. *P<0.05, **P<0.01, ***P<0.001, ****P<0.0001; ns, not significant.

FIG. 13. The NMR spectrum of different crosslinkers (i) NPC linker; (ii) PEG NPC linker and (iii) 4-arm-PEG NPC linker. CDCl₃, Chemical shift δ (ppm): 8.26 (d, NO₂—C—CH—, peak a), 7.37 (d, —CH—C—OOC—, peak b), 4.57 (m, —COO—CH₂—CH₂—S, peak c), 3.08, (m, —COO—CH₂—CH₂—S, peak d).

FIG. 14. The FT-IR spectrum of different crosslinkers and their precursors. FI-IR: ^{V}(C—O)=1767 cm⁻¹, ^{V}(Ar—H)=1596 cm⁻¹, ^{V}(Ar—NO₂)=1519 cm⁻¹ and 1336 cm⁻¹, ^{V}(C—O)=1105 cm⁻¹.

FIG. 15. The UV-Vis spectrum of DNA, crosslinker, and side products (4-Nitrophenol) after reactions.

FIG. 16(a-b). (FIG. 16a) The UV-Vis spectrum of 4-nitrophenol at different concentrations; (FIG. 16b) The calibration curve of 4-nitrophenol.

FIG. 17(a-f). Detailed results of DNA condensation via RECAST (FIG. 17a) The crosslinking density measurements of different linkers with the reaction time; (FIG. 17b) The size of DNA after reacting with different linkers in different time points; (FIG. 17c) The DNA surface potential changes with reaction time after reacting with different linkers; (FIG. 17d) the DLS tests, and (FIG. 17e) the zeta potential of different PEI/original DNA or PEI/b-DNA complexes at different N/P ratios; (FIG. 17f) Gel electrophoresis tests of original DNA, NPC condensed DNA, PEG₂₀₀₀ NPC condensed DNA and 4-arm PEG NPC condensed DNA in 0.5% TAE gel. All plots show mean±SEM unless otherwise mentioned, n>3 per group.

FIG. 18(a-d). Green fluorescence protein (GFP) expression images of HEK cells by different PEI/DNA complexes at (FIG. 18a) N/P of 10 and (FIG. 18b) 15, (i) original DNA, and condensed DNA via (ii) NPC linker, (iii) 4-arm PEG NPC linker, (iv) PEG₁₀₀₀ NPC linker, (v) PEG₂₀₀₀ NPC linker, (vi) PEG₃₄₀₀ NPC linker, (vii) PEG₅₀₀₀ NPC linker, scale bar: 100 μm. The corresponding fluorescence intensity quantification analysis at (FIG. 18c) N/P of 10 and (FIG. 18d) 15. n=4 per group, one-way ANOVA.

FIG. 19(a-c). The cell viability tests of different PEI/DNA complexes at (FIG. 19a) N/P 5, (FIG. 19b) N/P 10 and (FIG. 19c) N/P 15. n>3 per group, one-way ANOVA. All plots show mean±SEM unless otherwise mentioned. *P<0.05, **P<0.01, ***P<0.001, ****P<0.0001; ns, not significant.

FIG. 20(a-c). Green fluorescence protein (GFP) expression images of HEK cells (6.2 Kbp plasmids) by various Lipofectamine 2000/DNA complexes at (FIG. 20a) ratio 1:1, (FIG. 20b) ratio 2:1, and (FIG. 20c) ratio 3:1, (i) original DNA, and condensed DNA via (ii) NPC linker, (iii) 4-arm PEG NPC linker, (iv) PEG₁₀₀₀ NPC linker, (v) PEG₂₀₀₀ NPC linker, (vi) PEG₃₄₀₀ NPC linker, (vii) PEG₅₀₀₀ NPC linker, scale bar: 100 μm.

FIG. 21(a-f). The corresponding fluorescence intensity quantification analysis of Lipofectamine 2000/DNA complexes at (FIG. 21a) ratio 1:1, (FIG. 21b) ratio 2:1 and (FIG. 21c) ratio 3:1, and the flow cytometry quantification analysis at (FIG. 21d) ratio 1:1, (FIG. 21e) ratio 2:1 and (FIG. 21f) ratio 3:1. n>3 per group, one-way ANOVA. All plots show mean±SEM unless otherwise mentioned. *P<0.05, **P<0.01, ***P<0.001, ****P<0.0001; ns, not significant.

FIG. 22. The flow cytometry quantification analysis of Lipofectamine 2000/DNA complexes at ratio 1:1, 2:1 and ratio 3:1. n>3 per group, one-way ANOVA. All plots show mean±SEM unless otherwise mentioned. *P<0.05, **P<0.01, ***P<0.001, ****P<0.0001; ns, not significant.

FIG. 23(a-b). Green fluorescence protein (GFP) expression images (11.6 Kbp plasmids) of HEK cells by different PEI/DNA complexes at (FIG. 23a) N/P of 10 and (FIG. 23b) N/P of 15, (i) original DNA, and condensed DNA via (ii) NPC linker, (iii) 4-arm PEG NPC linker, (iv) PEG₁₀₀₀ NPC linker, (v) PEG₂₀₀₀ NPC linker, (vi) PEG₃₄₀₀ NPC linker, (vii) PEG₅₀₀₀ NPC linker, scale bar: 100 μm.

FIG. 24(a-b). The corresponding fluorescence images quantification analysis of various PEI/DNA complexes at (FIG. 24a) N/P of 10, and (FIG. 24b) N/P of 15. The positive cells were counted by Image J. (n>3 per group, one-way ANOVA. All plots show mean±SEM unless otherwise mentioned. *P<0.05, **P<0.01, ***P<0.001, ****P<0.0001; ns, not significant.

FIG. 25(a-c). Green fluorescence protein (GFP) expression images of HEK cells (11.6 Kbp plasmids) by various Lipofectamine 2000/DNA complexes at (FIG. 25a) ratio 1:1, (FIG. 25b) ratio 1:2 and (FIG. 25c) ratio 3:1. (i) original DNA, and condensed DNA via (ii) NPC linker, (iii) 4-arm PEG NPC linker, (iv) PEG₁₀₀₀ NPC linker, (v) PEG₂₀₀₀ NPC linker, (vi) PEG₃₄₀₀ NPC linker, (vii) PEG₅₀₀₀ NPC linker, scale bar: 100 μm.

FIG. 26(a-c). The corresponding fluorescence images quantification analysis of various Lipofectamine 2000/DNA complexes at (FIG. 26a) ratio 1:1; (FIG. 26b) ratio 2:1 and (FIG. 26c) ratio 3:1. The positive cells were counted by Image J. n>3 per group, one-way ANOVA. All plots show mean±SEM unless otherwise mentioned. *P<0.05, **P<0.01, ***P<0.001, ****P<0.0001; ns, not significant.

FIG. 27. The DLS tests of condensed DNA after incubation for 0 and 6 h in 10 mM GSH solution.

FIG. 28(a-b). Live mice were imaged using IVIS after tail vein injection (FIG. 28a) 12 h and (FIG. 28b) 24 h. Female C57BL/6 mice (n=5) received tail-vein injections of vivo-Jet PEI nanoparticles formulated with 2 versions of b-DNA (PEG 2K and PEG 5K) and original DNA encoding for firefly luciferase at a dose of 1.5 mg/kg.

### V. DETAILED DESCRIPTION

48. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims that follow, reference will be made to a number of terms which shall be defined to have the following meanings:

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

“Optional” or “optionally” means that the subsequently described event or circumstance may or may not occur, and that the description includes instances where said event or circumstance occurs and instances where it does not.

The term “biocompatible” generally refers to a material and any metabolites or degradation products thereof that are generally non-toxic to the recipient and do not cause significant adverse effects to the subject.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, reagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, reagents, conjugates, active metabolites, isomers, fragments, analogs, etc.

A “control” is an alternative subject or sample used in an experiment for comparison purposes.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

A “nucleic acid” is a deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered sugar moieties, such as a locked nucleic acid (LNA).

A “nucleotide” is a fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

The term “polynucleotide” refers to a single or double stranded polymer composed of nucleotide monomers (DNA or RNA).

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Sequence identity” is defined as the similarity between two nucleic acid sequences is expressed in terms of the similarity between the sequences, otherwise referred to as sequence identity. Sequence identity is frequently measured in terms of percentage identity, similarity, or homology; a higher percentage identity indicates a higher degree of sequence similarity. The NCBI Basic Local Alignment Search Tool (BLAST), Altschul et al, J. Mol. Biol. 215:403-10, 1990, is available from several sources, including the National Center for Biotechnology Information (NCBI, Bethesda, MD), for use in connection with the sequence analysis programs blastp, blastn, blastx, tblastn and tblastx. It can be accessed through the NCBI website. A description of how to determine sequence identity using this program is also available on the website. When less than the entire sequence is being compared for sequence identity, homologs will typically possess at least 75% sequence identity over short windows of 10-20 amino acids, and can possess sequence identities of at least 85% or at least 90% or 95% depending on their similarity to the reference sequence. Methods for determining sequence identity over such short windows are described on the NCBI website. These sequence identity ranges are provided for guidance only; it is entirely possible that strongly significant homologs could be obtained that fall outside of the ranges provided.

A “subject” is any mammal, such as humans, non-human primates, pigs, sheep, horses, dogs, cats, cows, rodents and the like. In two non-limiting examples, a subject is a human subject or a murine subject.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset, during early onset, or after an established development of a disorder or symptoms thereof. Prophylactic administration can occur for several days to years prior to the manifestation of symptoms of a disorder.

“Therapeutically effective amount” or “therapeutically effective dose” of a composition (e.g., a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Nanocomposite

In some aspects, disclosed herein is a nanocomposite comprising a cationic vector and a chemically modified nucleic acid that comprises at least one nucleobase that is linked to a chemical crosslinker via a reversible covalent bond. Reversible covalent bonds are able to change their bond arrangement via reversible reaction triggered by external stimuli such as heating, light, pH, enzymes, small molecules, while retaining the stability of irreversible covalent arrangement in the absence of the stimuli. The chemically modified nucleic acid can comprise at least one nucleobase linked to a chemical crosslinker via a reversible covalent bond. In some embodiments, the reversible covalent bond is a carbamate bond, an imides bond, or an amides bond. The glutathione-responsive disulfide moiety can be placed at the β-position of the carbamate to respond to the highly reductive intracellular microenvironment, as the cleavage of disulfide bond under glutathione can further contribute to the carbamate cleavage for the release of the nucleic acid. In some embodiments, the chemical crosslinker is a p-nitrophenylcarbonate crosslinker (e.g., disulfides (ethane-2,1-diyl)bis(4-nitrophenyl) bis(carbonate) (NPC)). In some embodiments, the chemical crosslinker is PEGylated (e.g., a PEGylated NPC crosslinker). In some embodiments, the PEGylated NPC crosslinker has a molecular weight of about 500 g/mol to 10,000 g/mol (for example, 1000 g/mol, 2000 g/mol, 3000 g/mol, 3400 g/mol, or 5000 g/mol). In some embodiments, the PEGylated NPC crosslinker has a molecular weight of about 500 g/mol to 3,000 g/mol, about 1000 g/mol to 5,000 g/mol, about 1000 g/mol to 10,000 g/mol, or about 3000 g/mol to 7000 g/mol. In some embodiments, the PEGylated NPC crosslinker is a 4-arm PEGylated NPC crosslinker that comprises four NPC groups. In some embodiments, the PEGylated NPC crosslinker is an 8-arm PEGylated NPC crosslinker that comprises eight NPC groups. In some embodiments, the PEGylated NPC crosslinker is a 16-arm PEGylated NPC crosslinker that comprises sixteen NPC groups. In some embodiments, the PEGylated NPC crosslinker is a dendrimer.

In some embodiments, the chemically modified nucleic acid forms a bundle that is less than about 50 nm in size, less than about 100 nm in size, less than about 150 nm in size, less than about 200 nm in size, less than about 250 nm in size, less than about 300 nm in size, less than about 400 nm in size, less than about 500 nm in size, or less than about 600 nm in size. In some embodiments, the chemically modified nucleic acid forms a bundle that is less than about 150 nm in size. In some embodiments, the chemically modified nucleic acid forms a bundle that has a diameter from about 1 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle has a diameter from about 100 nm to about 250 nm. In some embodiments, the chemically modified nucleic acid forms a bundle that has a diameter from about 150 nm to about 175 nm. In some embodiments, the chemically modified nucleic acid forms a bundle that has a diameter from about 135 nm to about 175 nm. The bundled nucleic acid is about 10 times, 9 times, 8 times, 7 times, 6 times, 5 times, 4 times, 3 times, 2.5 times, 2 times, or 1.5 times smaller in size than a non-bundled nucleic acid. Methods of making these bundles are described below. The bundles can have any shape but are generally spherical in shape.

In some embodiments, the nucleic acid is a DNA or an RNA. In some embodiments, the nucleic acid is at least about 5000 base pairs (bp) in length (for example, at least about 6000 bp in length, at least about 8000 bp in length, at least about 10,000 bp in length, at least about 15,000 bp in length, at least about 20,000 bp in length, at least about 25,000 bp in length, at least about 30,000 bp in length, at least about 40,000 bp in length, at least about 50,000 bp in length, or at least about 100,000 bp in length).

In some embodiments, the cationic vector is a cationic polymer or a cationic liposome.

In some embodiments, the cationic vector is a cationic liposome composed of cationic lipids. Cationic lipids have a head group with positive charge. Examples of cationic lipids include, but are not limited to, 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), 2,3-dioleyloxy-N-[2-(sperminecarboxamido)ethyl]-N,N-dimethyl-1-propanaminium trifluoroacetate (DOSPA), or 2-(((((3S,8S,9S,10R,13R,14S, 17R)-10,13-dimethyl-17-((R)-6-methylheptan-2-yl)-2,3,4,7,8,9,10,11,12,13,14, 15, 16, 17-tetradecahydro-1H-cyclopenta[a]phenanthren-3-yl)oxy) carbonyl)amino)-N,N-bis(2-hydroxyethyl)-N-methylethan-1-aminium bromide (BHEM-Cholesterol).

In some embodiments, the cationic vector comprises a cationic polymer. Examples of cationic polymers include, but are not limited to, a polyethylene imine (PEI) polymer, a poly-β-amino ester (PBAEs) polymer, a branched poly(amino ester) (PAE) polymer, a polymethacrylate polymer, a cyclodextrin (cd)-based polymer, a chitosan-based polymer, a dendrimer-based polymer, a polysaccharide-based cationic polymer, or a polydisulfide amine polymer. In some embodiments, the PEI polymer is Glycerol 1,3-diglycerolate diacrylate, 1,3-Butanediol diacrylate, 1, 6-Hexanediol diacrylate, Ethylene glycol diacrylate, Triethylene glycol diacrylate, Ethylene glycol (n) diacrylate, Propylene glycol glycerolate diacrylate, Tri (propylene glycol) glycerolate diacrylate, Tri (propylene glycol) diacrylate, Poly(propylene glycol) diacrylate, Trimethylolpropane ethoxylate methyl ether diacrylate, Glycerol propoxylate triacrylate, Trimethylol propane ethoxylate triacrylate, Tricycle [5.2.1.0]decane-dimethanol diacrylate, 1,3,5-triacryloyl hexahydro-1,3,5-triazine, Di(trimethylol propane) tetraacrylate, Pentaerythritol tetraacrylate, Dipentaerythritol penta-/hexa acrylate, Bisphenol A ethoxylate diacrylate, Bisphenol A propoxylate diacrylate, Bisphenol A propoxylate glycerolate diacrylate, Trimethylolpropane ethoxylate triacrylate, Bisphenol F ethoxylate di acrylate, or Pentaerythrfol propoxylate triacrylate. In some embodiments, the cationic polymer is a polyethylene imine (PEI) polymer. In some embodiments, the PEI polymer is a branched polyethylenimine cationic polymer that is about 25,000 MW in size (i.e., bPEI₂₅ₖ).

Also disclosed herein is a pharmaceutical composition comprising the nanocomposite of any preceding aspect and a pharmaceutically acceptable carrier.

Also disclosed herein are kits that are drawn to reagents that can be used in practicing the methods disclosed herein. In some aspects, disclosed herein is a kit for introducing a nucleic acid into a cell, said kit comprising the nanocomposite of any preceding aspect. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### C. Methods of Producing Nanocomposites

In some aspects, disclosed herein is a method of producing a nanocomposite for delivering a nucleic acid, said method comprising providing a chemical crosslinker; contacting the nucleic acid with the chemical crosslinker thereby creating a chemically modified nucleic acid that comprises at least one nucleobase that is linked to a chemical crosslinker via a reversible covalent bond; and contacting the chemically modified nucleic acid with a cationic vector thereby packaging the chemically modified nucleic acid into the cationic vector to produce the nanocomposite. The chemically modified nucleic acids (i.e., crosslinked) are condensed into bundled nucleic acid (b-DNA or b-RNA) with smaller size via a reversible covalent self-assembly process. The product produced by this method is referred to herein as, “bundled nucleic acid” or “bDNA.”

In some embodiments, the reversible covalent bond is a carbamate bond, an imides bond, or an amides bond. The reversible covalent bond can link the chemical crosslinker to an amine of the at least one nucleobase. In some embodiments, the chemical crosslinker is a p-nitrophenylcarbonate crosslinker (e.g., disulfides (ethane-2,1-diyl)bis(4-nitrophenyl) bis(carbonate) (NPC)). In some embodiments, the chemical crosslinker is PEGylated (e.g., a PEGylated NPC crosslinker). In some embodiments, the PEGylated NPC crosslinker has a molecular weight of about 500 g/mol to 10,000 g/mol (for example, 1000 g/mol, 2000 g/mol, 3000 g/mol, 3400 g/mol, or 5000 g/mol). In some embodiments, the PEGylated NPC crosslinker has a molecular weight of about 500 g/mol to 3,000 g/mol, about 1000 g/mol to 5,000 g/mol, about 1000 g/mol to 10,000 g/mol, or about 3000 g/mol to 7000 g/mol. In some embodiments, the PEGylated NPC crosslinker is a 4-arm PEGylated NPC crosslinker that comprises four NPC groups.

In some embodiments, the nucleic acid and the chemical crosslinker are in contact at a ratio in weight of about 1:1, 1:2. 1:3, 1:4, 1:5, 1:6, 1:7, 1:8, 1:9, or 1:10.

In some embodiments, the chemically modified nucleic acid forms a bundle that is less than about 50 nm in size, less than about 100 nm in size, less than about 150 nm in size, less than about 200 nm in size, less than about 250 nm in size, less than about 300 nm in size, less than about 400 nm in size, less than about 500 nm in size, or less than about 600 nm in size. In some embodiments, the chemically modified nucleic acid forms a bundle that is less than about 150 nm in size. In some embodiments, the chemically modified nucleic acid forms a bundle that has a diameter from about 1 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle has a diameter from about 100 nm to about 250 nm. In some embodiments, the chemically modified nucleic acid forms a bundle that has a diameter from about 150 nm to about 175 nm. In some embodiments, the chemically modified nucleic acid forms a bundle that has a diameter from about 135 nm to about 175 nm. The bundled nucleic acid is about 10 times, 9 times, 8 times, 7 times, 6 times, 5 times, 4 times, 3 times, 2.5 times, 2 times, or 1.5 times smaller in size than a non-bundled nucleic acid. The bundles can have any shape but are generally spherical in shape.

In some embodiments, the nucleic acid is a DNA or an RNA. In some embodiments, the nucleic acid is at least about 5000 bp in length (for example, at least about 6000 bp in length, at least about 8000 bp in length, at least about 10,000 bp in length, at least about 15,000 bp in length, at least about 20,000 bp in length, at least about 25,000 bp in length, at least about 30,000 bp in length, at least about 40,000 bp in length, at least about 50,000 bp in length, or at least about 100,000 bp in length.

In some embodiments, the cationic vector is a cationic polymer or a cationic liposome. In some embodiments, the cationic vector is a cationic liposome. Accordingly, in some embodiments, step c) of the method of producing a nanocomposite disclosed herein comprises contacting the cationic liposome (e.g., lipofectamine) and the chemically modified nucleic acid at a weight ratio of about 3:1, 2:1, 1:1, 1:2, 1:3, 1:5, 1:8, or 1:10. In some embodiment, the cationic liposome (e.g., lipofectamine) and the chemically modified nucleic acid are in contact at a weight ratio of about 1:1, 1:2, or 1:3.

In some embodiments, the cationic vector is a cationic polymer. In some embodiments, a cationic polymer is a polyethylenimine (PEI) cationic polymer. In some embodiments, the PEI polymer is a branched polyethylenimine cationic polymer that is about 25,000 g/mol in size (i.e., bPEI₂₅ₖ, linear formula is H(NHCH₂CH₂)ₙNH₂). Accordingly, in some embodiments, step b) of the method of producing a nanocomposite disclosed herein comprises contacting the cationic polymer and the chemically modified nucleic acid at a nitrogen/phosphorus (N/P) ratio of about 1:1, 2.5:1, 5:1, 7.5:1, 10:1, 12.5:1, 15:1, 20:1, 25:1, 30:1, 40:1, or 50:1. In some embodiments, the cationic polymer and the chemically modified nucleic acid is in contact at a nitrogen/phosphorus (N/P) ratio of about 5:1, 10:1, or 15:1, or from about 1:1 to about 50:1, from about 2:1 to about 20:1, from about 2:1 to about 7:1, from about 2:1 to about 12:1, from about 2:1 to about 17:1, from about 5:1 to about 13:1, from about 7:1 to about 13:1, from about 12:1 to about 17:1, from about 5:1 to about 10:1, from about 10:1 to about 15:1, or from about 15:1 to about 20:1.

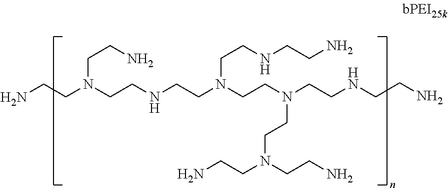

Also disclosed are nanocomposites produced by the methods disclosed herein.

In some embodiments, the chemically modified nucleic acid forms a bundle that is less than about 50 nm in size, less than about 100 nm in size, less than about 150 nm in size, less than about 200 nm in size, less than about 250 nm in size, less than about 300 nm in size, less than about 400 nm in size, less than about 500 nm in size, or less than about 600 nm in size. In some embodiments, the chemically modified nucleic acid forms a bundle that is less than about 150 nm in size. In some embodiments, the chemically modified nucleic acid forms a bundle that has a diameter from about 1 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle has a diameter from about 100 nm to about 250 nm. In some embodiments, the chemically modified nucleic acid forms a bundle that has a diameter from about 150 nm to about 175 nm. In some embodiments, the chemically modified nucleic acid forms a bundle that has a diameter from about 135 nm to about 175 nm. The bundled nucleic acid is about 10 times, 9 times, 8 times, 7 times, 6 times, 5 times, 4 times, 3 times, 2.5 times, 2 times, or 1.5 times smaller in size than a non-bundled nucleic acid. The bundles can have any shape but are generally spherical in shape.

In some embodiments, the nucleic acid is a DNA or an RNA. In some embodiments, the nucleic acid is at least about 5000 bp in length (for example, at least about 6000 bp in length, at least about 8000 bp in length, at least about 10,000 bp in length, at least about 15,000 bp in length, at least about 20,000 bp in length, at least about 25,000 bp in length, at least about 30,000 bp in length, at least about 40,000 bp in length, at least about 50,000 bp in length, or at least about 100,000 bp in length).

### D. Methods of Modulating Nucleic Acids in Cells

In some aspects, disclosed herein is a method of introducing a nucleic acid into a cell, said method comprising providing the nanocomposite disclosed herein or the pharmaceutical composition disclosed herein, wherein the nanocomposite or the pharmaceutical composition comprises the nucleic acid; and contacting the nanocomposite with the cell thereby introducing nucleic acid into the cell.

In some embodiments, the cell is a mammalian cell. In some embodiments, the nucleic acid is a DNA or an RNA.

It is herein contemplated that the nanocomposites and pharmaceutical compositions can be used for disease treatments of genetic disorders by modulating levels of nucleic acids in cells in a subject in need.

Accordingly, in some aspects, disclosed herein is a method of preventing or treating a genetic disorder in a subject in need, said method comprising providing the nanocomposite disclosed herein or the pharmaceutical composition disclosed herein, wherein the nanocomposite or the pharmaceutical composition comprises the nucleic acid; and administering a therapeutically effective amount of the nanocomposite to the subject in need.

In some aspects, disclosed herein is a method of modulating a level of a nucleic acid in a cell in a subject in need, said method comprising providing the nanocomposite disclosed herein or the pharmaceutical composition disclosed herein, wherein the nanocomposite or the pharmaceutical composition comprises the nucleic acid; and administering a therapeutically effective amount of the nanocomposite to the subject in need.

As mentioned above, the methods and compositions disclosed herein can be used to treat or prevent a genetic disorder in a subject. By “treat” or “prevent” is meant that the severity of the disease is reduced or prevented by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, as compared to a control, or to a subject before treatment.

In some embodiments, the subject has a genetic disorder. In some embodiments, the genetic disorder is a cancer or neurological disease (e.g., Parkinson's disease, epilepsy, or autism). About 15% of people with Parkinson's disease have a family history of the condition, and family-linked cases can result from genetic mutations in a group of genes—LRRK2, PARK2, PARK7, PINK1, and/or the SNCA gene. Accordingly, in some embodiments, the administration of the nanocomposite increases the level of nucleic acid (e.g., DNA or RNA) of wide type LRRK2, PARK2, PARK7, PINK1, or SNCA in a subject having Parkinson's disease. Genetic predisposition combined with environmental conditions lead to epilepsy. About 30% to 40% of epilepsy is caused by genetic predisposition, including, for examples, mutations or other abnormalities in gene CDKL5, PCDH19, Ring chromosome 20, SCN8A related SLC2A1 (Glut1 Deficiency Syndrome), TBCK-related ID, Rett-MECP2, FOXG1, Dup 15q, SYNGAP1, KCNQ2, KCNQ3, STXBP1, SCNIA, SCN2A, ARX, CHD2, GRIN2A, PRRT2, KCNT1, CLN2, CLN genes, POLG1, Ring 14, STRADA, DNM1, PTEN, SLC25A22, SPTAN1, FOLR1, CACNAIA, HCN1, WWOX, GRIN2B, GABRA1, LGI1, MEF2C, SLC6A1, SLC13A5, ALDH7A1, CHRNA4, SMC1A, and/or GATOR1. Accordingly, in some embodiments, the administration of the nanocomposite increases the level of nucleic acid (e.g., DNA or RNA) of wide type CDKL5, PCDH19, SCN8A related SLC2A1 (Glut1 Deficiency Syndrome), TBCK-related ID, Rett-MECP2, FOXG1, Dup 15q, SYNGAP1, KCNQ2, KCNQ3, STXBP1, SCNIA, SCN2A, ARX, CHD2, GRIN2A, PRRT2, KCNT1, CLN2, CLN genes, POLG1, Ring 14, STRADA, DNM1, PTEN, SLC25A22, SPTAN1, FOLR1, CACNA1A, HCN1, WWOX, GRIN2B, GABRA1, LGI1, MEF2C, SLC6A1, SLC13A5, ALDH7A1, CHRNA4, SMCIA, and/or GATOR1 in a subject having epilepsy. In some embodiments, the genetic disorder is cystic fibrosis. In some embodiments, the genetic disorder is a genetic lung disease with the gene target of CFTR. Accordingly, in some embodiments, the administration of the nanocomposite increases the level of nucleic acid (e.g., DNA or RNA) of wide type CFTR in a subject having cystic fibrosis. In some embodiments, the genetic disorder is Alzheimer's disease. Accordingly, in some embodiments, the administration of the nanocomposite increases the level of nucleic acid (e.g., DNA or RNA) of wide type APOE4, APOE2, and/or MAPT in a subject having Alzheimer's disease. In some embodiments, the genetic disorder is autism. Accordingly, in some embodiments, the administration of the nanocomposite increases the level of nucleic acid (e.g., DNA or RNA) of wide type UBE3A, MECP2, FMR, TSC1, TSC2, SHANK3, NLGN4X, NRXN1A, SHANK2, SCN2A, CHD8, and/or SYNGAP1 in a subject having autism.

The compositions or systems disclosed herein can be used to treat genetic disorders by, for example, delivering the normal or wide-type nucleic acid (e.g., DNA or RNA) into cells or delivering constructs, vectors, or plasmids that encode a gene-editing machinery, e.g., CRISPR, into cells. Accordingly, in some examples, the nanocomposite disclosed herein comprises a chemically modified nucleic acid encoding a gene-editing machinery (including, for example, CRISPR-Cas9 system).

In some embodiments, the administration of the nanocomposite increases the level of the nucleic acid in the subject. By “increase the level” is meant the amount of nucleic acid is increased in the subject by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, for example, or by 2, 3, 4, 5, 6, 7, 8, 9, or 10 fold or more. This increase is compared to a control to which the nanocomposite has not been administered, or to the subject before treatment.

In another aspect, the nanocomposite comprises a nucleic acid that decreases or encodes a suppressor that decreases a level of a protein in a cell. By “decrease the level” is meant the amount of protein is decreased in the subject by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, for example, or by 2, 3, 4, 5, 6, 7, 8, 9, or 10 fold or more. This increase is compared to a control to which the nanocomposite has not been administered, or to the subject before treatment.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day. Dosages above or below the range cited above may be administered to the individual patient if desired.

### 1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

In some embodiments, the compositions disclosed herein are administered intravenously. This delivery system can circulate in blood in stealthy style due to the introduction of PEG, and can mainly accumulate at liver and kidney. In some embodiments, the compositions disclosed herein can be used for liver or kidney targeting gene therapy.

The compositions may be administered orally, parenterally (e.g., intravenously), by intramuscular injection, by intraperitoneal injection, transdermally, extracorporeally, topically or the like, including topical intranasal administration or administration by inhalant. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### VI. EXAMPLE

### Example 1. Materials and Methods

Material: 2-hydroxyethyl disulfide, 4-nitrophenyl chloroformate, p-nitrophenol, branched polyethylenimine (PEI, 25K), reductive glutathione (GSH), Poly-L-ornithine (PLO, MW: 30-70 kD), Tween-20, 5-fluoro-2′-deoxyuridine and paraformaldehyde (PFA) were purchased from Sigma-Aldrich. The amino function polyethylene glycol (NH₂-PEG-NH₂, 1K, 2K, 3.4K, and 5K) and 4-arm amino function polyethylene glycol (4-arm-PEG-NH₂, 2K) were purchased from Creative PEGWorks. The cellTiter-Blue reagent was purchased from Promega. Lipofectamine 2000 reagent, DAPI, Triton X-100, and general cell culture reagents were ordered from Thermo Fisher Scientific. A nucleic acid labeling Kit (Cy5) was ordered from Mirus. The in-vivo JetPEI and JetOPTIMUS gene delivery reagents were ordered from Polyplus Transfection. D-Luciferin, Potassium Salt was ordered from GoldBio. NeuN primary antibody (ab 177487) was purchased from AbCam. Secondary antibody Alexa 594 (R37117) was ordered from Thermo Fisher Scientific. Hoechst 33342 was purchased from AAT Bioquest. pCMV-GFP was a gift from Connie Cepko (Addgene plasmid #11153; n2t.net/addgene: 11153; RRID: Addgene_11153). pcDNA3-EGFP was a gift from Doug Golenbock (Addgene plasmid #13031; n2t.net/addgene: 13031; RRID: Addgene_13031). pcDNA3.1-dCas9-dMQ1-EGFP was a gift from Margaret Goodell (Addgene plasmid #89637; n2t.net/addgene: 89637; RRID: Addgene_89637). Luciferase-pcDNA3 was purchased from Addgene (Addgene plasmid #18964: www.addgene.org/18964/).

Characterization: Nuclear Magnetic Resonance (NMR) spectra were measured by the Aligent-400 NMR spectrometer. The size and zeta potential of DNA were measured by dynamic light scattering (DLS) using Zetasizer Nano-ZS from Malvern Instruments. The UV-Vis spectra were determined by Eppendorf Biospectrometer. The FT-IR spectra were monitored via Infrared Spectrometer. The fluorescence images were obtained by Leica DMi8 fluorescence microscope. The confocal laser scanning microscopy (CLSM) images were obtained via ZEISS LSM 980.

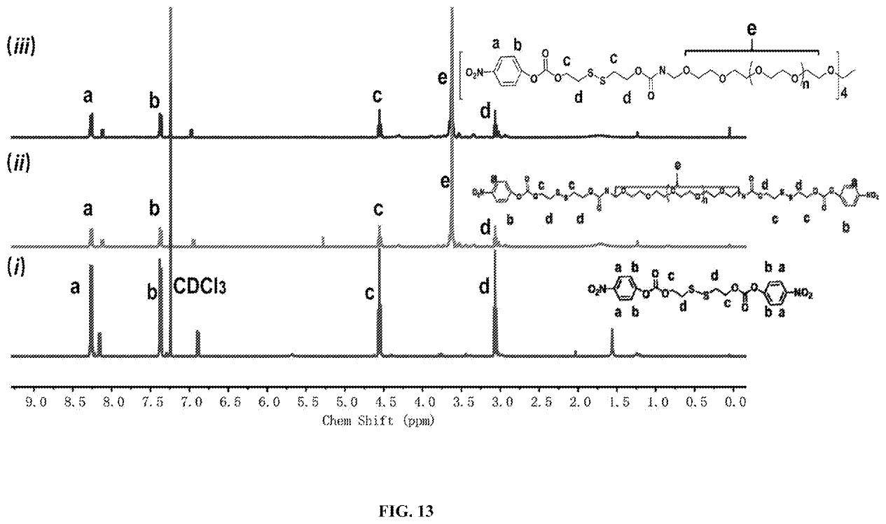

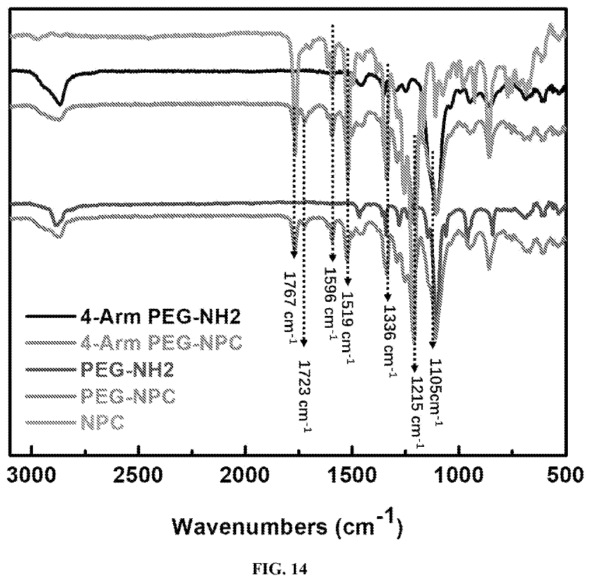

Synthesis of disulfides (ethane-2,1-diyl)bis(4-nitrophenyl) bis(carbonate) crosslinker (NPC). 2-hydroxyethyl disulfide (4 mmol, 0.616 g) and 4-nitrophenyl chloroformate (8.5 mmol, 1.713 g) were dissolved in 30 mL dichloromethane (CH₂Cl₂). Then the triethylamine (8.5 mmol, 0.86 g) was added into 10 mL CH₂Cl₂, and added dropwise into the reaction solution at an ice water bath. After reacting for 2 h, the reaction solution was put at room temperature and continued to react 24 h. Finally, the white solids were removed via vacuum filtration, and the filtrate was dried by rotary evaporation. The raw product was collected and purified via silica column chromatography with the mixture of petroleum ether/ethyl acetate (1:1, v/v). We obtained the final product after removing the solution, with yields of 61.9%, as shown in FIG. 13 (i). ¹H NMR (400 MHZ, CDCl₃): δ (ppm) 8.26 (d, NO₂—C—CH—), 7.37 (d, —CH—C—OOC—), 4.57 (m, —COO—CH₂—CH₂—S), 3.08, (m, —COO—CH₂—CH₂—S). FI-IR: ^{V}(C═O)=1767 cm⁻¹, ^{V}(Ar—H)=1596 cm⁻¹, ^{V}(Ar—NO₂)=1519 cm⁻¹ and 1336 cm⁻¹, ^{V}(C—O)=1105 cm⁻¹, as shown in FIG. 14.

Synthesis of NPC groups functional polyethylene glycol crosslinkers (PEG-NPC) and 4-arm PEG crosslinker (4-arm PEG-NPC). Briefly, the NPC and NH₂-PEG-NH₂ (4-arm-PEG-NH₂) were added into 10 mL of CH₂Cl₂ with a ratio of 1:2, and the solution was stirred for 24 h at room temperature. After that, the solution was washed with saturated ammonium chloride solution several times until the organic solution became colorless. The organic solution was collected and dried by anhydrous sodium sulfate. The final yellowish solid was obtained after removing the solution via rotary evaporation with a yield of 86.7%, as shown in FIG. 13 (ii) and (iii). 1H NMR (400 MHZ, CDCl₃): δ (ppm) 8.25 (d, NO₂—C—CH—), 7.36 (d, —CH—C—OOC—), 4.56 (m, —COO—CH₂—CH₂—S), 3.68 (m, —O—CH₂—CH₂—O—) 3.09, (m, —COO—CH₂—CH₂—S). FI-IR: ^{V}(C═O)=1767 cm-1, ^{V}(OCO—NH—)=1723 cm-1, ^{V}(Ar—H)=1596 cm-1, ^{V}(Ar—NO₂)=1519 cm⁻¹ and 1336 cm⁻¹, ^{V}(Ar—NO₂)=1519 cm⁻¹, as shown in FIG. 14.

Preparation of condensed DNA. A covalent self-assembly method is developed to condense the DNA plasmid to a smaller size. Briefly, 1 mg DNA and 4 mg crosslinkers were added to the PBS. After adjusting the solution pH to 8.5, the reaction mixture was stirred for 24 h at room temperature. Finally, the mixture was collected and washed with PBS at least 5 times using Amicon Ultra Centrifugal Filters MWCO 3 kDa to remove the crosslinkers. The final volume of condensed DNA solution was adjusted to 0.5 mL with PBS (pH 7.4), and the DNA concentration was measured via UV-Vis spectrum.

Gel electrophoresis tests. The gel electrophoresis tests were performed in 0.5 wt % agarose gel. The original DNA and condensed DNA were mixed with loading buffer and electrophoresed in Tris-acetate-EDTA (TAE) buffer (1×) at 150 V for 50 min. The DNA bands were observed via Gel Doc XR image analyzer.

Nanocomposites size and zeta potential tests. Commercial reagent branched PEI (25 K) was chosen as a model delivery system. For the branched PEI system, the genes were mixed with PEI at different N/P of 5, 10, 15. For example, to prepare the DLS test samples (N/P of 10), 5 μg DNA was first added into 50 μL PBS, and then 6.5 μg PEI was added. The mixture was vortexed for 10 s and stored at room temperature for 30 min for the DLS tests.

The in vitro release of DNA. 1 mg condensed DNA was added into 1 mL 10 mM GSH PBS solution, and the solution was incubated at 37° C. At fixed time intervals, 0.1 mL solution was withdrawn for the DLS tests. 4 h later, the reaction solution was collected and washed with PBS at least 5 times using Amicon Ultra Centrifugal Filters MWCO 3 kDa to purify DNA. The final volume of the released DNA solution was adjusted to 0.5 ml with PBS, and the DNA concentration was monitored by UV-Vis spectrum. To detect whether the structure of released DNA is damaged compared with the original DNA, 10 μg original DNA and released DNA were used for gene sequence tests.

DNA labeled via Cy5 dye. To evaluate the nucleus targeting efficiency of original DNA and condensed DNA, the DNA was labeled fluorescence dye via Nucleic acid labeling Kit (Cy5). Briefly, 10 μL DNA solution (original DNA or condensed DNA, 1 mg/mL), 5 μL label IT® Reagent, 5 μL 10× Labeling Buffer A and 30 μL DNsae-, RNase-free water were added into the tube, and the mixture was incubated at 37° C. for 1 h. After that, the samples were purified using G50 microspin purification columns to obtain the final solution. The solution was lyophilized, and 10 μL PBS was added to dissolve the solid to obtain the solution (the DNA concentration was 1 mg/mL).

In vitro gene transfection. The human embryonic kidney cell line 293T (HEK-293T) was cultured in Dulbecco's Modified Eagle Medium (DMEM) containing 10% FBS. Firstly, the HEK-293T were seeded in a 24-well plate and cultured until 70-80% confluence. Then DNA nanocomposites were prepared with 1 μg DNA used for each well. For the commercial transfection reagents, PEI was utilized at N/P of 5, 10, and 15, and Lipofectamine 2000 was utilized at a w/w ratio of 1:1. 1:2 and 1:3. Before adding the nanocomposites, the culture medium was replaced by fresh DMEM. Then, the DNA nanocomposites were added into the cells with 1 μg DNA each well. After incubation for 6 h, the culture medium was replaced by fresh DMEM containing 10% FBS. 48 h later, the cells were collected for gene transfection evaluations via fluorescence microscope and flow cytometry.

Embryonic primary cortical neuron cell culture. Controlled-breeding was performed to generate timed-pregnant WT mice to be sacrificed after 15.5 days of pregnancy to retrieve E15.5-day old embryonic pups. Cortex regions of brains from pups were dissected and pooled in dissection buffer (Minimum Essential Medium, 10% heat-inactivated horse serum, 0.5 mM Glutamine), then dissociated with trypsin containing 0.25% EDTA. Cells were plated at an ideal density in 24 or 48-well plates precoated with 200 μg/mL solution of Poly-L-ornithine (MW: 30-70 kDa) for 1 h at 37° C. and 7% CO₂ then washed twice with sterile DI water and PBS. Cells were plated in culture media consisting of Neurobasal Medium, 2% B27 Supplement, and 0.5 mM Glutamine. After seeding for 30 minutes, the media was replaced, and cells were incubated at 37° C. and 7% CO₂. After 3 days, 5-fluoro-2′-deoxyuridine glial inhibitor diluted to 0.1 mM in 0.5 well-volume culture media was added to cells.

In vitro gene transfection in primary neurons. DIV3 primary embryonic cortical neurons plated at a suitable density in a 48 well plate was used. Prior to treatment, half of culture media was removed and placed into a new plate to be incubated at 37° C. and 7% CO₂. Wells were treated with DNA nanocomposites consisting of 0.4 μg of pCMV-GFP DNA and 0.4 μL of jetOPTIMUS® diluted to 50 μL with Opti-MEM. Solutions were vortexed and incubated at room temperature for 10 min before adding dropwise to wells. After 3 hours, the removed culture media was returned to the corresponding wells. After 24 hours, cells were washed, fixed and stained. Briefly, culture media was removed, and cells were washed with DPBS and fixed with 4% w/v PFA in PBS for 30 min at 4° C. Fixed cells were washed twice with DPBS before permeabilization with 0.3% w/v Triton X-100 for 10 min. Cells were washed with DPBS and then blocked with a solution of 10% v/v heat-inactivated goat serum for 1 h. Following blocking, samples were incubated overnight in a primary antibody solution containing rabbit anti-NeuN (1:800 dilution). Following, the primary antibody solution was removed, and cells were washed 3 times with 0.1% v/v Tween20 solution in DPBS and then incubated for in the secondary antibody solution containing goat anti-rabbit conjugated to Alexa 594 (1:200 dilution) and Hoechst 33342 (1:5000 dilution) in 5% goat serum in DPBS. Lastly, cells were washed 3 times with DPBS. Fluorescent microscopy was performed on stained cells at 20× magnification and images were processed and analyzed via ImageJ script for frequency of colocalization of DAPI+, NeuN+ and GFP+ signals to determine transfection efficiency.

Cell viability tests. Interior 60-wells of 96-plate were coated with 10 μg/mL solution of PLO (Poly-L-Ornithine) at 50 μL/well 37° C. for 1 h. The solution was removed and washed twice with 100 μL of PBS. The HEK293T cells were seeded in interior 60 wells of a coated 96-well plate at a density of 1.75×10⁴ cells/well and cultured for 24 hrs until 70% confluency in DMEM containing 10% FBS. Then DNA nanocomposites were prepared with 0.2 μg DNA used for each well. For the commercial transfection reagents, PEI was utilized at N/P of 5, 10, and 15 (n=4). Before adding the nanocomposites, the culture medium was replaced by 100 μL fresh DMEM. Then, 20 μL DNA nanocomposites were added into the cells, with 2 μg DNA each well. After incubation for 6 h, the culture medium was replaced with 100 μL of fresh DMEM containing 10% FBS. 48 h later, 20 μL Cell-Titer blue was added and shaken for 10 seconds. Cells were incubated at 37° C. for 4 h and then analyzed via fluorimetry (560 nm ex/590 nm em).

Nucleus delivery efficiency evaluation. The HEK-293T were seeded in a 24-well plate with coverslips and cultured until 70-80% confluence. The culture medium was withdrawn and replaced via fresh DMEM. After 1 h incubation, fresh opti-MEM containing PEI/Cy5 labeled original DNA and PEI/Cy5 labeled condensed DNA were added into each well, respectively. Cy5 labeled DNA was added at 1 μg/well, and the N/P was 10. 6 h later, the cells were washed with PBS three times and fixed with 4% paraformaldehyde solution for 15 min at room temperature. After washing with PBS three times, the nucleus was stained with DAPI (10 μg/mL) for 15 min. The prepared samples were observed with confocal laser scanning microscopy (CLSM) to evaluate the nucleus delivery efficiency.

In vivo gene delivery. All procedures involving animals were conducted in accordance with the National Institutes of Health Guide for the Care and Use of Laboratory Animals following protocols approved by the University of Texas, Austin Institutional Animal Care and Use Committee. 8-10 weeks old (20-25 g) Female C57BL/6 mice (n=5 per group) received i.v. tail-vein injections of in vivo-JetPEI nanocomposites formulated with 2 versions of b-DNA (PEG 2K and PEG 5K) and original DNA encoding for firefly luciferase (Luciferase-pcDNA3) at a dose of 1.5 mg/kg. Briefly before usage, 150 μg DNA (PEG₂k, PEG₅k, original) were diluted to 500 μL in a 5% glucose solution to final concentration of 300 μg/mL. For each sample, 24 μL of in vivo-JETPEI (N/P 8) was diluted to 500 μL in a 5% glucose solution and added to the nucleic acid solutions. Solutions were vortexed for 10 seconds and incubated at room temperature for 15 min prior to usage. Mice (n=5 per group) were restrained and administered 200 μL of solutions via tail vein before being returned to their cage. Prior to in vivo bioluminescence imaging, mice were anesthetized with 2% isoflurane and hair on underside was removed by shaving and a chemical depilatory (Nair) to reduce signal interference. For in vivo bioluminescence imaging (IVIS, PerkinElmer) at 6, 12 and 24 hours post injection, mice were anesthetized using 2.5% isoflurane and delivered i.p. 200 μL of 15 mg/mL D-Luciferin solution in DPBS. After 10 min, mice groups were imaged (binning: 4, F/stop: 1, Exposure time: 1 min, FOV:D, smoothing: 3×3) for bioluminescence and brightfield. Total flux and radiance of signal was determined by ROI selection over mice abdominal space. Following the last time point (24 hours), organs were harvested from each animal and imaged using the same settings (FOV=B). Total flux and radiance of signal was determined by ROI selection around each organ.

### Example 2. Reversible, Covalent DNA Condensation Approach Via Chemical Linkers for Enhanced Gene Delivery

Viral approaches have been widely applied for gene delivery, but this approach suffers from limited gene packing size, host immune response, and high manufacturing costs. Non-viral gene delivery has been explored as an alternative approach but generally suffers from lower efficiency. In particular, efficient nucleus delivery of DNA has been the Achilles' heel of non-viral approach. Current non-viral DNA condensation systems are mainly based on electrostatic interaction between cationic polymer/liposomes and anionic DNA plasmids. However, the electrostatic interaction is too weak to keep DNA condensed to diffuse through the cytoplasm and transport into the nucleus for gene transcription initiation. Here, this study shows a two-stage condensation approach to achieve efficient cytoplasm diffusion and nuclear transport. Firstly, chemical linkers were utilized to crosslink DNA plasmids via a reversible covalent bond to form smaller-sized bundled DNA (b-DNA). Then, the b-DNA was packaged into cationic vectors to further condense b-DNA and enable efficient gene delivery to the nucleus. This study demonstrates clear improvements in gene transfection efficiency with b-DNA, including using DNA plasmid with size up to 11.6 kbp. Furthermore, the improved gene transfection efficiency with b-DNA was also observed in primary cultured neurons. Finally, a clear improvement in lung-selective gene transfection efficiency in vivo by this two-stage condensation approach was observed following intravenous administration. This reversible covalent assembly strategy demonstrates substantial value of non-viral gene delivery for clinical therapeutic applications.

Genetic engineering is a promising tool for both fundamental understanding of biology and treatment of diseases at the genomic level. Over the past decades, it has been utilized in optogenetics to study the neural circuit, in chimeric antigen receptor (CAR) based cell cancer therapy, and in genetic modification in inherited diseases and neurodegenerative diseases. Viral vectors, such as adeno-associated vectors (AAV), have become a widely exploited vehicle to express exogenous DNA into target cells due to their high transfection efficiency. For example, AAV vectors used to treat congenital blindness have been approved by Food and Drug Administration (FDA) since 2017. However, several setbacks of viral vectors severely limited the gene therapy in clinical trials, including immunogenicity, carcinogenesis, limited DNA packaging capacity (DNA<5 Kbp) and high production cost. Non-viral vectors have great potential to address many of the limitations of viral vectors, particularly concerning safety, packaging capacity and costs. Most ionizable cationic and permanently cationic polymers, such as poly(β-amino ester) s polymers, polypeptides, dendrimer polymers, polyethylenimine derivatives and gold nanoparticles have been developed to deliver genes, including mRNA, DNA and siRNA, or CRISPR editing system. Recently, lipid nanoparticles-based mRNA vaccines have been successfully developed and widely applied against the COVID-19.

While significant developments have been achieved, the gene delivery efficiency of non-viral vectors for plasmid DNA delivery is not sufficient for clinical needs. A common observation with non-viral vectors is their generally low transfection efficiency, particularly in post-mitotic cells. Especially, since DNA complexes readily dissociate and lose transfection ability upon dilution. In addition, the high doses of cationic vectors are often associated with cytotoxicity. Lastly, gene transcription is generally difficult to initiate when the DNA plasmid size is larger than 10 Kbp, due to the inefficient condensation and nucleus transport of cationic non-viral vectors. Fundamentally, efficient gene transfer requires not only the entry of DNA from the extracellular surface of the cell into the cytoplasm but also delivery across the nuclear envelope and into the nucleus before any transcription can initiate. In the current non-viral vector strategy, larger-sized DNA plasmid is mainly condensed and packaged into cationic nanoparticles via electrostatic interaction in high vector concentrations and then released into the cytoplasm after escaping from endosomes via proton sponge effect. While viral vectors can enable direct nuclear entry of DNA plasmids, in non-viral vectors most of the released DNA plasmids are unable to sufficiently diffuse through the viscous cytoplasm and enter the nucleus via ˜60 nm diameter nuclear pore complexes channels to initiate transcription.

To address the nuclear entry challenges, DNA minivector, linear DNA, or nucleus targeting proteins have all been explored with improved efficiency in nuclear transport. However, the minivector and linear DNA strategy required breakage of DNA plasmid backbone, which decreases the stability of DNA and thus increases the risk of wrong genome integration. On the other hand, nucleus targeting proteins, such as associating nuclear localization signals (NLSs), were used to join the linear DNA to achieve enhanced nucleus transport but only exhibited minimal improvement of gene expression efficiency for large DNA plasmids. Other than these direct DNA editing strategies, researchers have also developed post-crosslinking approaches to shrink the DNA complexes size through reversible intermolecular crosslinking of cationic carriers to achieve nuclear delivery of genes. However, this crosslinking can downregulate the proton sponge effect and retard endosomal escape due to the decrease in the density of ionizable cationic charges in these carriers. Additionally, DNA is not able to be effectively released from these crosslinked carriers due to the high crosslinking density, thus decreasing the transfection efficiency.

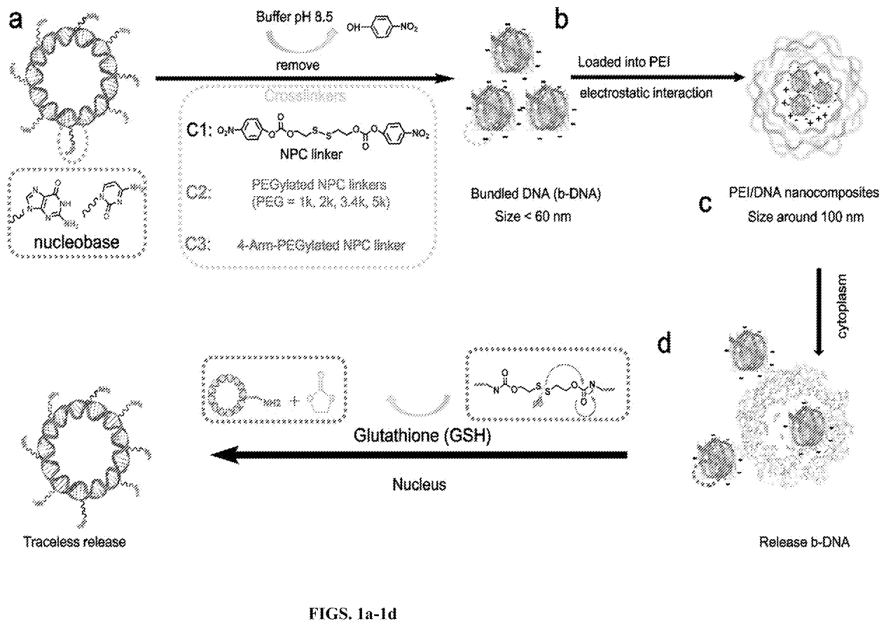

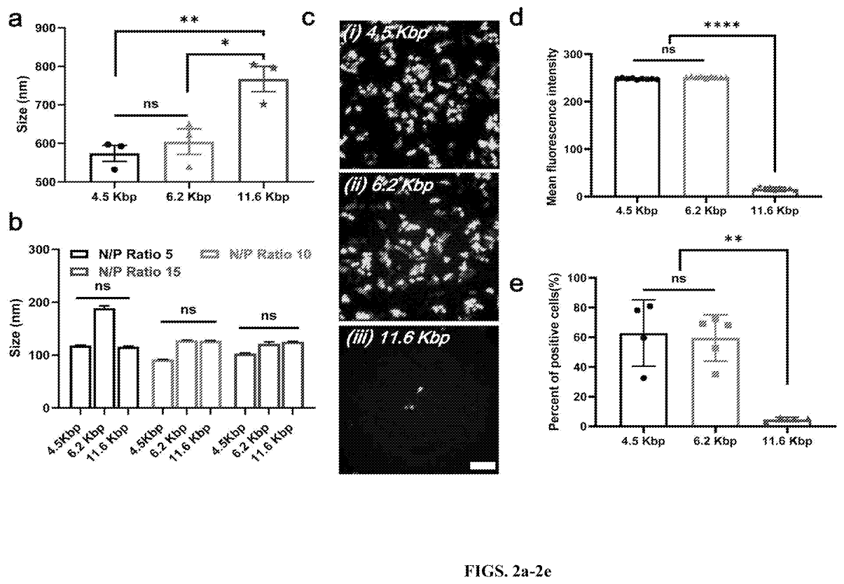

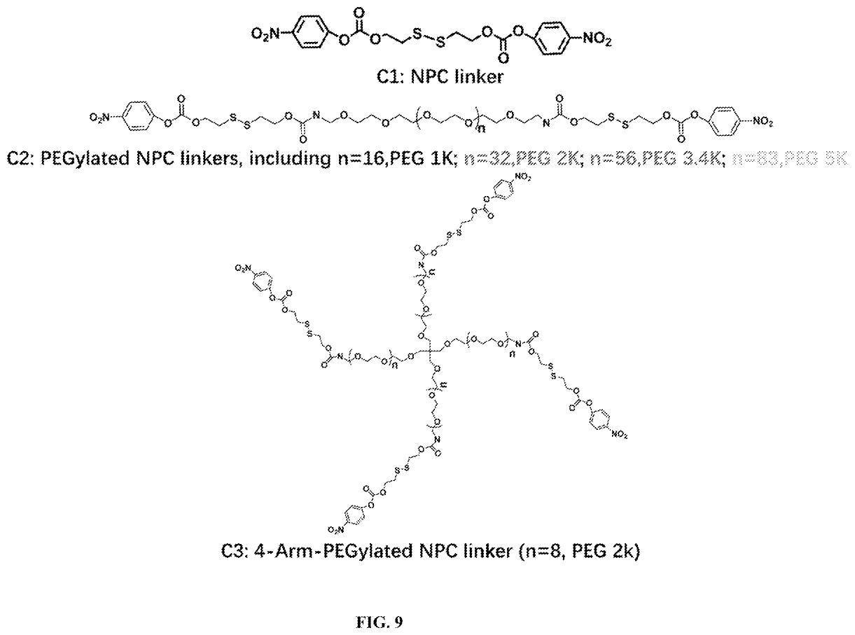

This work developed a two-stage condensation approach to achieve efficient gene delivery, and which is named the strategy Reversible Covalent Assembly Strategy (RECAST). As shown in FIG. 1, the DNA plasmids react with a crosslinker (FIG. 9) to be firstly condensed into bundled DNA (b-DNA) with a smaller size (<60 nm). Then, these b-DNA plasmids are compressed again by a cationic delivery system via electrostatic interaction to form complexes with the size of around 100 nm. As a result, the b-DNA with a smaller size diffuse in viscous cytoplasm more rapidly than the original DNA and cross nucleopores into the nucleus. After diffusing into the nucleus, reductive cleavage of the crosslinkers results in the disassembly of b-DNA to tracelessly release the original DNA for effective gene transcription. Improved transfection efficiency has been achieved by our RECAST, including with DNA plasmid size of 11.6 kbp and in post-mitotic primary neurons. In addition, significant improvement in lung-selective gene transfection efficiency has been observed with this RECAST method following intravenous administration in mice, which demonstrates the substantial value of this RECAST for clinical therapeutic applications.

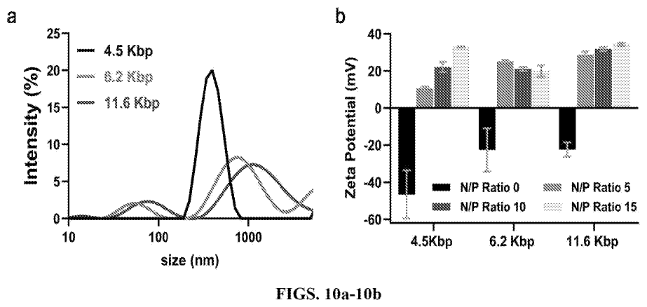

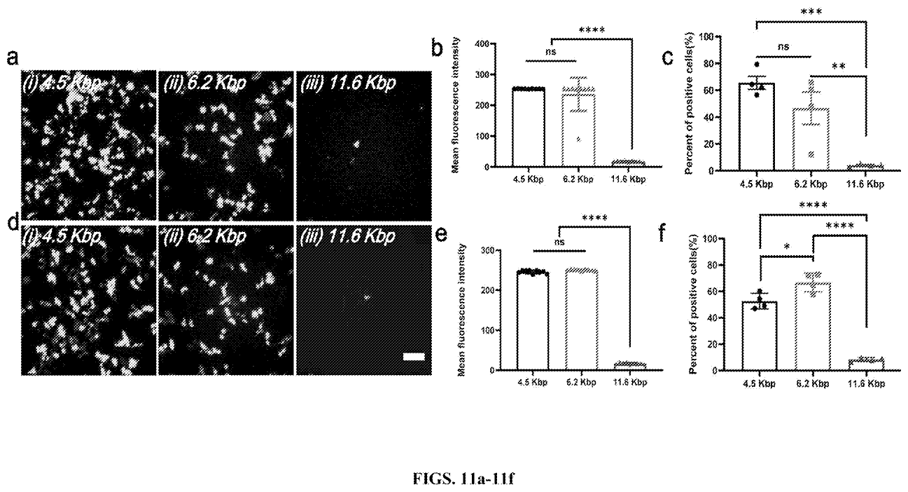

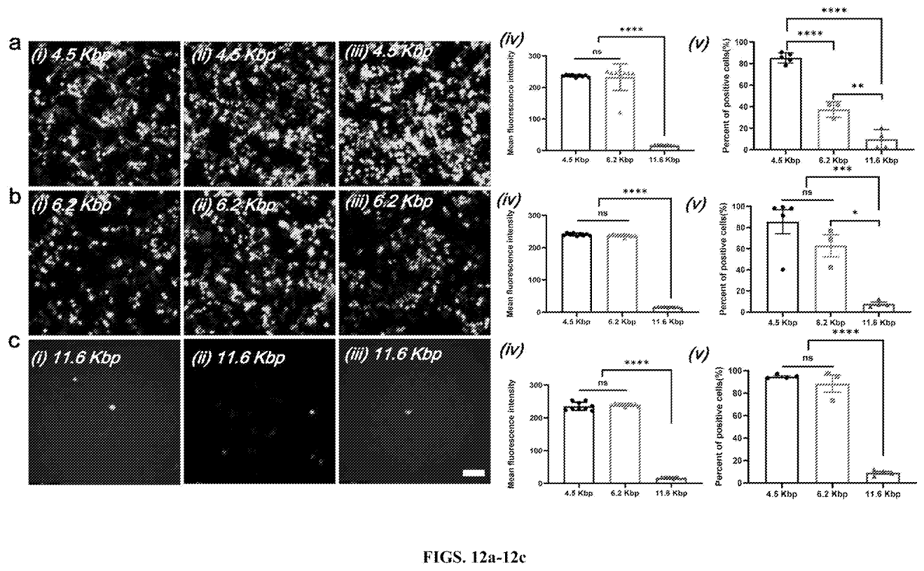

The hydration radius of DNA for gene transfection. Previous research has determined that the relative diffusion coefficient of DNA in the cytoplasm dramatically decreased with increased DNA size in the cytoplasm, thus retarding the gene transfection efficiency. Hideyoshi et al. have also demonstrated that gene expression is negatively associated with DNA size via microinjecting the DNA into the cytoplasm. However, the relationship between the hydration radius and molecular size of DNAs remains poorly understood but is important for understanding the nuclear transport of DNAs in non-viral delivery. To better understand the DNA molecular size-hydration radius relationship, we utilized various green fluorescence protein (GFP) encoded plasmids with different sizes. This study determined the DNA hydration radius of three sizes of plasmids (4.5 Kbp, 6.2 Kbp, and 11.6 Kbp) by dynamic light scattering, as shown in FIG. 2a and FIG. 10a. The data showed that the hydration radius of plasmids exhibited a positive correlation with the number of base pairs, which is consistent with Flory-Huggins theory. The hydration diameter of the DNA at 4.5 Kbp was ˜570 nm and increased to ˜770 nm when molecular size increased to 11.6 Kbp, which is a magnitude larger than nuclear pore complex channels (˜60 nm). To evaluate the effect of hydration size of plasmids on gene transfection efficiency, commercial branched polyethylenimine (bPEI₂₅ₖ) cationic polymer was chosen as a model delivery system to condense and load plasmids at Nitrogen to Phosphate (N/P) of 5, 10, and 15. As shown in FIG. 2b and FIG. 10b, these PEI/DNA complexes have similar size and surface potential but significantly reduced gene transfection efficiency of GFP expression (FIGS. 2c, 2d, and 11 from 4.5 Kbp plasmids to 11.6 Kbp plasmids. Flow cytometry was also used to determine the transfection efficiency, where the positive cell percentage decreased with the increased plasmid size with 70% transfection efficiency of 4.5 Kbp plasmids compared to less than 5% HEK 293T cells successfully transfected with the 11.6 Kbp plasmids, as shown in FIG. 2e. Of note, similar results were also observed in cationic lipids when the delivery system was switched to commercial Lipofectamine 2000 as shown in FIG. 12. The reason for the reduced transfection efficiency in large DNA plasmids can be that the DNA released in the cytoplasm can recover to the original hydration radius after releasing from the cationic vectors, preventing them from efficiently diffusing to the nucleus and entering the nucleus through nuclear pore complexes channels. This reason is supported by the previous observations of reduced DNA diffusion speed in the cytoplasm with increased DNA sizes. If the diffusion time is extended, the DNA can even experience degradation in the cytoplasm (half-time for DNA degradation is 50-90 min in the cytoplasm). Reducing DNA hydration radius with this RECAST strategy can enhance diffusion through the cytoplasm and the transport of DNA plasmids into the nucleus, thus improving gene delivery efficiency.

Design of the crosslinker for DNA condensation via covalent assembly strategy. Recently, gene intercalation via x-x stacking has been developed to crosslink DNA gel for siRNA delivery. In addition, a reversible protein crosslink strategy via covalent bonds have also been developed to achieve efficient intracellular protein delivery. However, until now, it remained poorly understood how to design the reversible chemical conjugation method to condense the DNA to achieve enhanced nucleus delivery. Nucleobase chemistry that uses the amines as reactive sites has been developed for DNA labeling, bioconjugations, and crosslinking over the past decades. To achieve effective crosslinking and traceless release of DNA, p-nitrophenylcarbonate functional crosslinkers were designed to react with amines in nucleobases to condense the DNA via carbamate bonds. The GSH responsive disulfide moiety is placed at the β-position of the carbamate to respond to the highly reductive intracellular microenvironment, as the cleavage of disulfide bond under GSH further contributes to the carbamate cleavage for traceless release of DNA. The detail of different crosslinkers is shown in FIG. 1 and FIG. 9 in supporting information. The crosslinker disulfides (ethane-2,1-diyl)bis(4-nitrophenyl) bis(carbonate) (NPC) was synthesized at first. Considering that the steric hindrance between p-nitrophenylcarbonate and amines would decrease NPC's reaction activity and hydrophobicity, the PEGylation of crosslinkers with different chain lengths and topological structure was also designed to control the reactivity and crosslinking density. As shown in FIGS. 12 and 13, the nuclear magnetic resonance (NMR) and Fourier-Transform Infrared Spectroscopy (FT-IR) spectra determined that the crosslinkers were successfully synthesized, with the clear characteristic peaks of products.

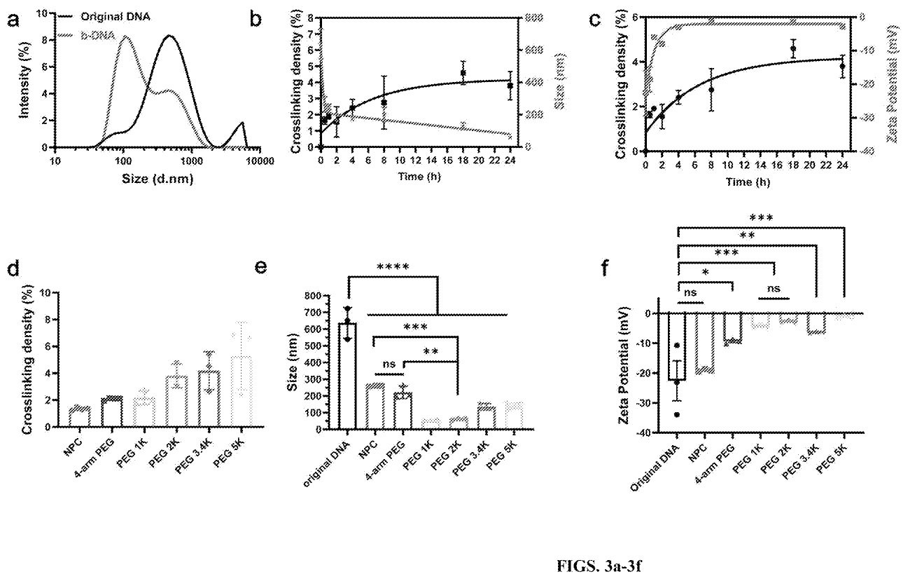

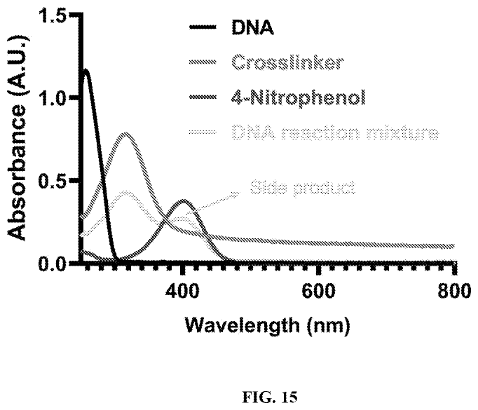

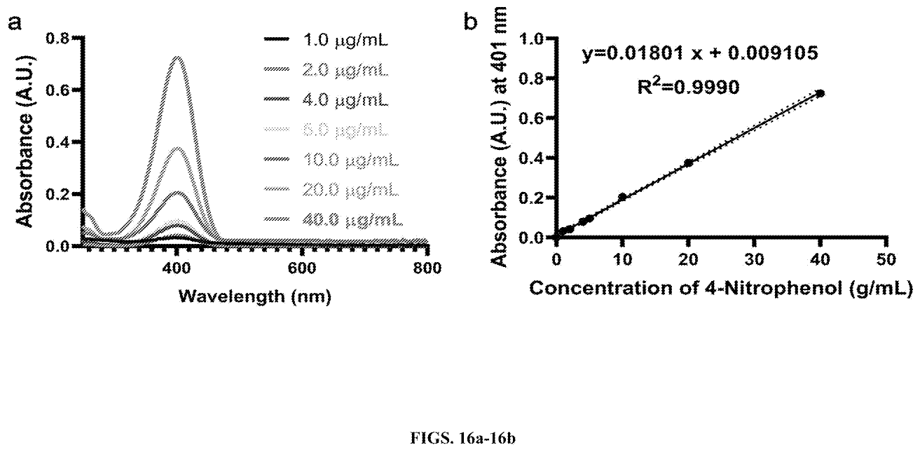

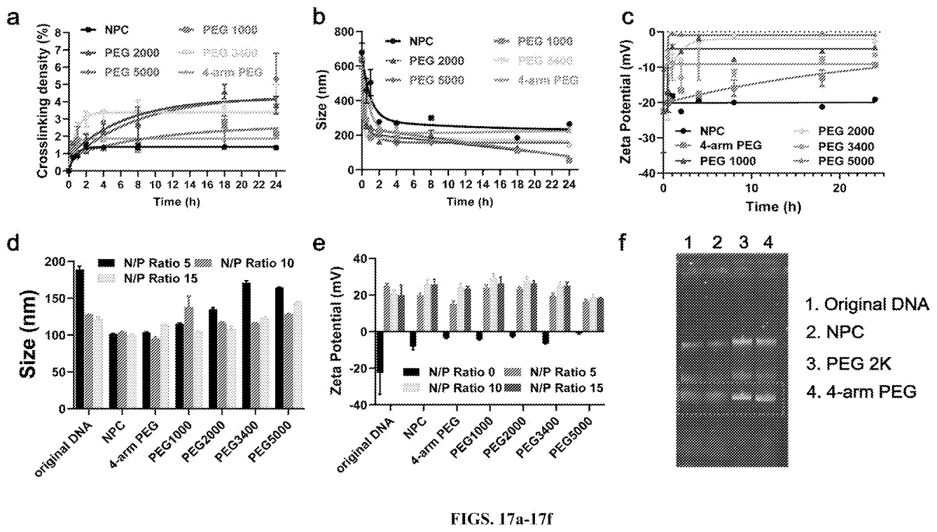

To test this strategy, 6.2 Kbp plasmid DNA was first chosen as a model system to react with the different crosslinkers. As shown in FIG. 15, the 4-nitrophenol side product was produced during the reaction and used as an indicator to evaluate the crosslinking process via calibration curve (FIG. 16). The crosslinking density increased as the reaction progressed and became almost saturated after reaction 24 h, while the DNA hydration size reduced with increased crosslinking density, as shown in FIG. 3a-3c and FIGS. 17a and 17b. The crosslinking density improved further with the introduction of PEG chains due to the reduced steric hindrance. In addition, the PEG chain length plays a primary role in controlling the reactivity between DNA and crosslinkers. (FIG. 3d). After reaction for 24 h, the DNA hydration size was condensed from 600 nm to below 300 nm for all of the chemical linkers, and the PEGylation crosslinker with molecular weight 2000 exhibited the better condensation efficiency, where the b-DNA size was around 50 nm (FIG. 3b, 3e and FIG. 17b). Notably, the surface potential of DNA increased with the integration of PEG chains since the PEG corona around the DNA can shield the negative surface potential of DNA (FIG. 3f and FIG. 17e). Meanwhile, the zeta potential depends on the PEG density and chain length. There are no remarkable surface potential differences between the original DNA and NPC condensed DNA due to the deficiency of PEG chains (FIG. 3f).

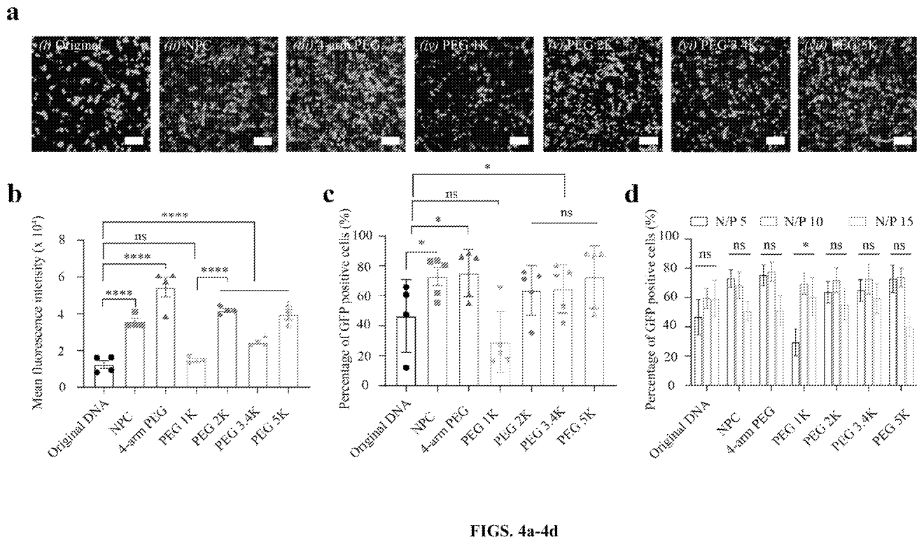

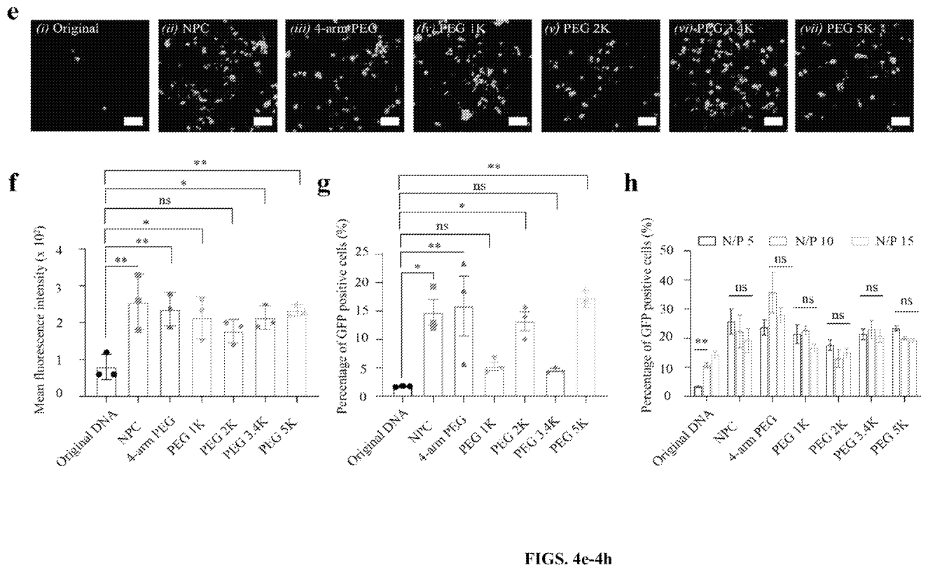

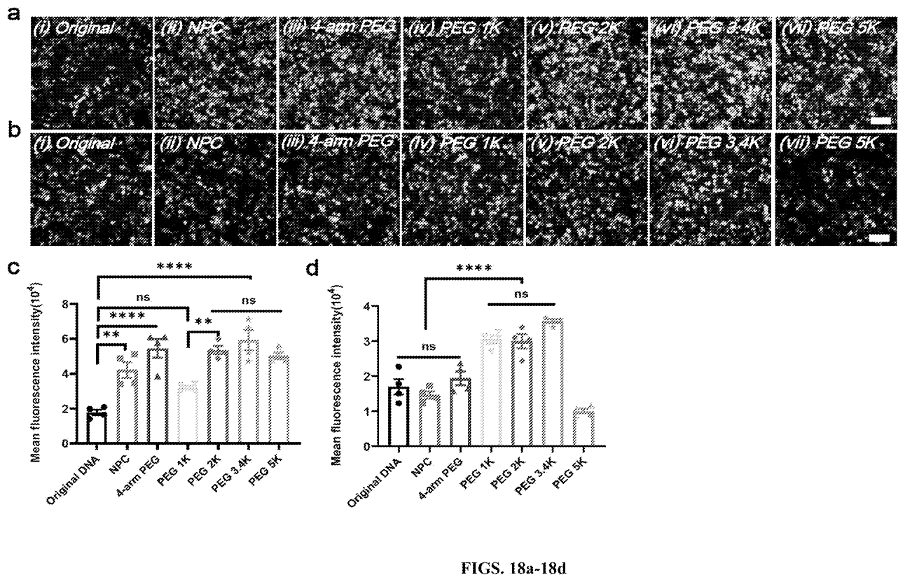

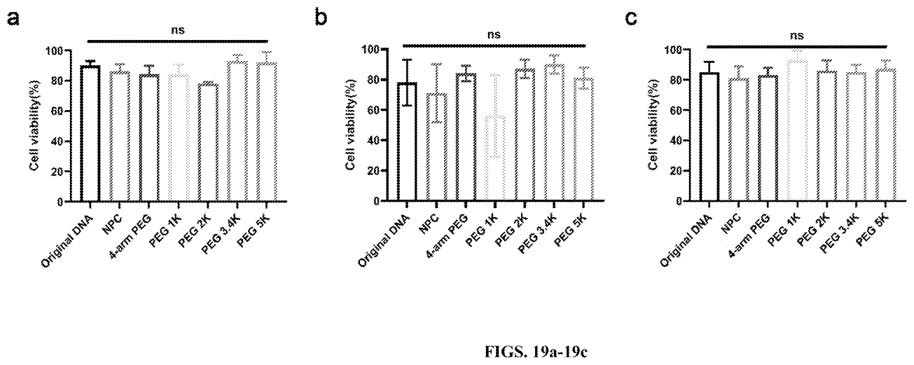

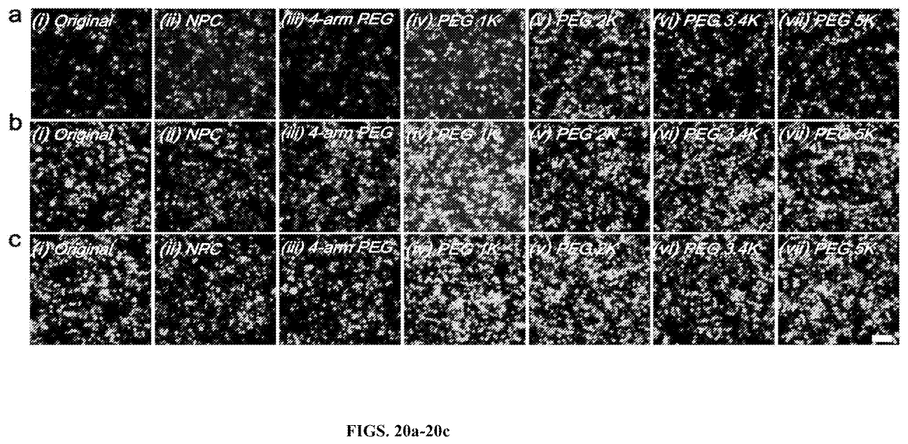

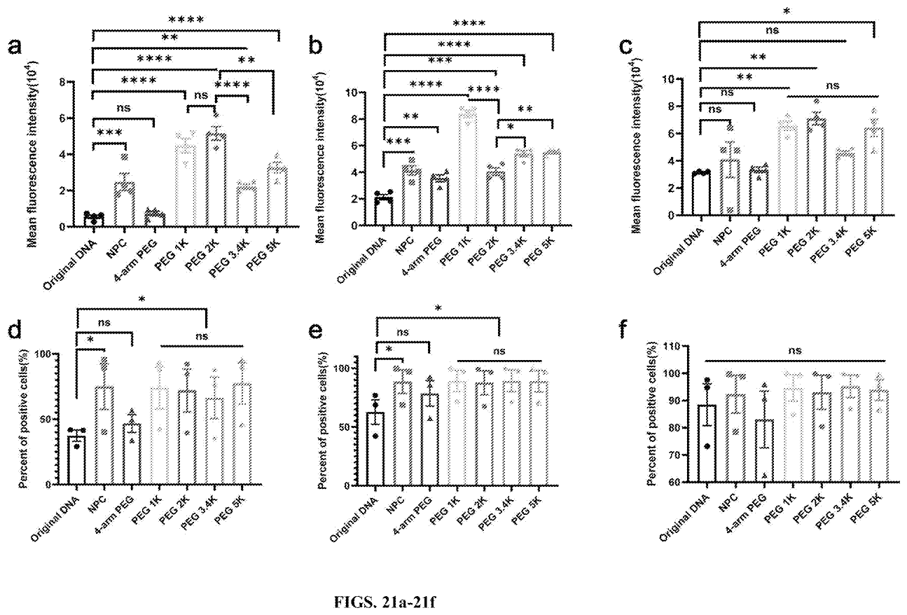

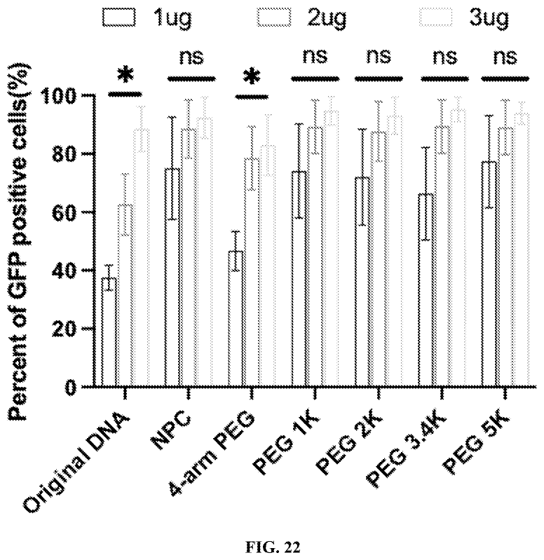

The gene transfection efficiency of b-DNA is enhanced in HEK cells. Next, to apply the RECAST strategy for transfection in HEK cells, commercial cationic polymer bPEI₂₅ₖ was used to condense the bundled DNA (b-DNA, 6.2 kbp) via electrostatic interaction. Although there were no differences in size and surface potential between PEI/b-DNA and PEI/original DNA complexes (FIGS. 17d and 17e), PEI/b-DNA complexes exhibited enhanced gene transfection efficiency, as shown in FIGS. 4a and 18. Here in FIG. 4a, the experiment firstly determined the transfection ability at low cationic polymer concentration, where the PEI/original DNA complexes showed weak GFP fluorescent signal at N/P of 5. Still, the GFP fluorescent signal intensity increased by at least a factor of 2 in PEI/b-DNA complexes (FIG. 4b). To support this, flow cytometry was also used to determine the transfection efficiency: the GFP+ cell percentage of PEI/original DNA was only around 50% but increased up to 80% in PEI/b-DNA complexes (FIG. 4c) at N/P of 5. Generally, cationic gene polyplexes usually exhibit improved gene transfection efficiency at higher cationic polymer concentrations but easily disassemble and lose transfection ability at low cationic polymer concentrations. However, cell toxicity generally increases with the increase in concentration of cationic polymers. To determine whether RECAST can effectively solve the contradiction between transfection efficiency and cell toxicity, this study also investigated bPEI₂₅ₖ gene transfection at different N/P for b-DNA (FIG. 4d and FIG. 18) and the relative cell viability (FIG. 19) in HEK cells. The results demonstrate that our RECAST exhibited high gene transfection efficiency even at low concentrations of cationic carriers (FIG. 4d and FIG. 18) with high biocompatibility. The improved transfection efficiency at lower N/P ratios can be because the two-stage condensation process allows for effective complexing of DNA at lower cationic polymer concentration. In addition, PEG_{1K} condensed DNA exhibited the smallest size, but PEG₅ₖ and 4-arm PEG condensed DNA showed better gene expression. The reason can be that the flexibility and conformation of DNA play a more crucial role in the nucleus transport and less restrictive PEG chain provided higher flexibility to adjust the morphology and fluidity of b-DNA to cross nuclear pore complex channels. To verify the universality of this strategy, another commercial transfection reagent, Lipofectamine 2000, was also applied for transfection, as shown in FIGS. 20 and 22. These results again demonstrated that Lipofectamine/b-DNA exhibited enhanced gene transfection efficiency than Lipofectamine/original DNA, from ˜35% to ˜75% GFP+ cells at lipofectamine/DNA ratios of 1:1 and from ˜65% to 90% GFP+ cells at lipofectamine/DNA ratios of 2:1.

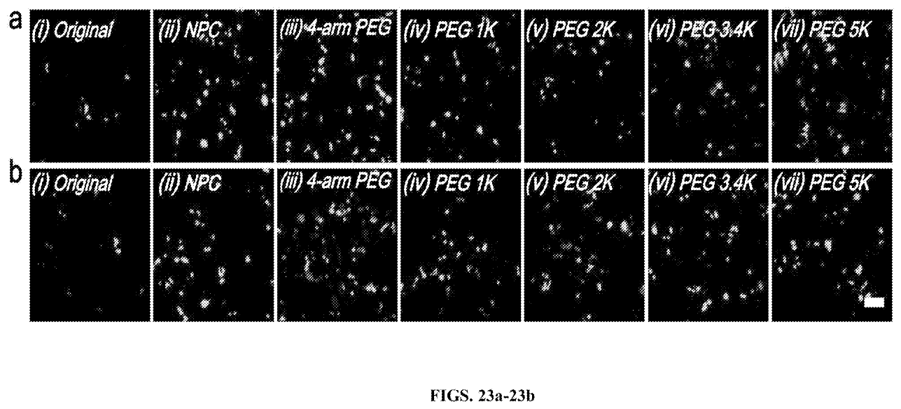

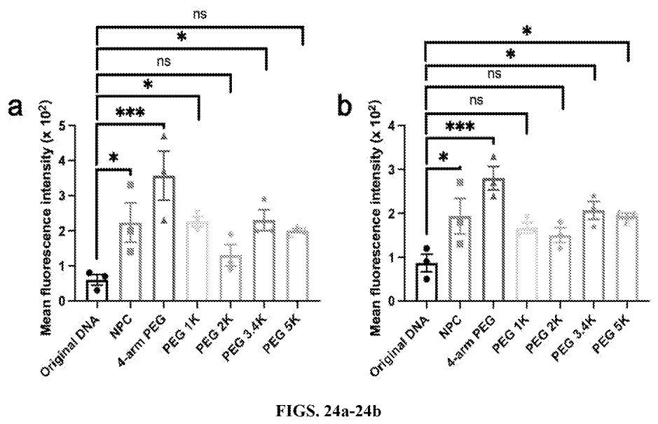

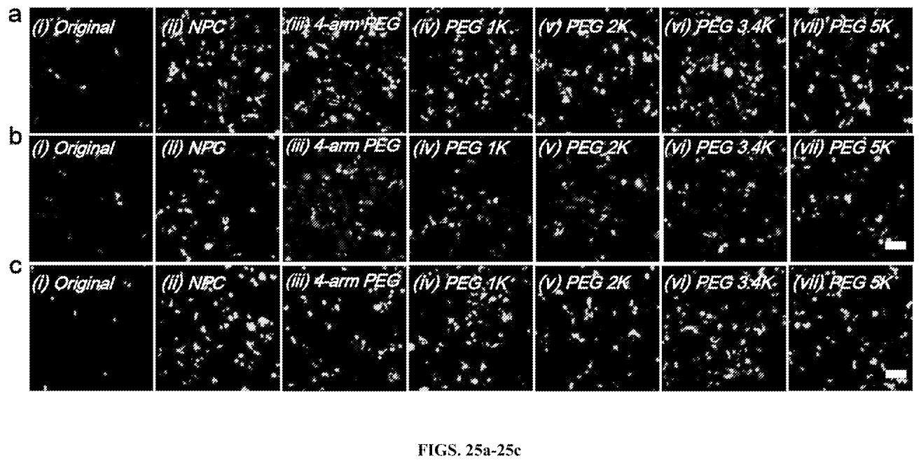

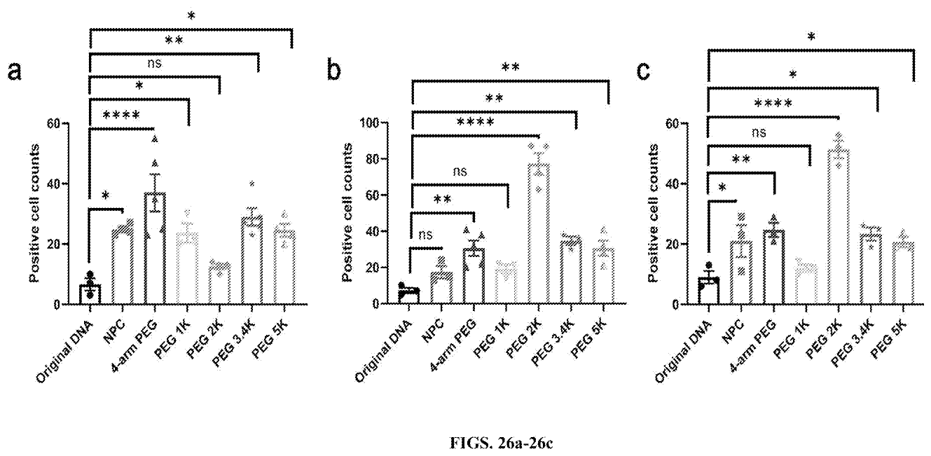

Generally, gene transcription is difficult for DNA plasmids larger than 7 kbp when using non-viral cationic delivery systems. For viral delivery, the use of lentivirus instead of AAV is also required for gene delivery of DNA plasmids of this size. This experiment again applied the RECAST approach for gene delivery with large DNA plasmids. Specifically, 11.6 kbp plasmids were reacted with crosslinkers to form b-DNA and packaged in bPEI₂₅ₖ to form complexes. As shown in FIGS. 4e-4g and FIGS. 23-24, the GFP signal increased in PEI/b-DNA complexes compared with PEI/original DNA at different doses, and the positive cell number increased by 2˜3 times compared to the PEI/original DNA transfection efficiency (FIGS. 4f-4g), indicating the enhanced gene transfection efficiency in PEI/b-DNA. Similar improvements were also observed when commercial Lipofectamine 2000 was used as the vector (FIGS. 25-26), which determined the strong commonality of the RECAST strategy. In particular, the flow cytometry data (FIG. 4h) demonstrated that the percentage of the GFP+ cells increased from ˜2% for PEI/original DNA to ˜15% PEI/b-DNA at N/P of 5. In comparison, the percentage of the GFP+ cells increased from ˜5% for Lipofectamine 2000/original DNA to >20% for Lipofectamine 2000/b-DNA at lipofectamine/DNA ratios of 2:1. These data demonstrated that this RECAST strategy is also efficient in improving gene transfection efficiency for large-size plasmids. In addition, this RECAST strategy exhibited vector dose-independent gene transfection performance (FIG. 4h), where the transfection efficiency was stable with the decrease of cationic carriers' concentration.

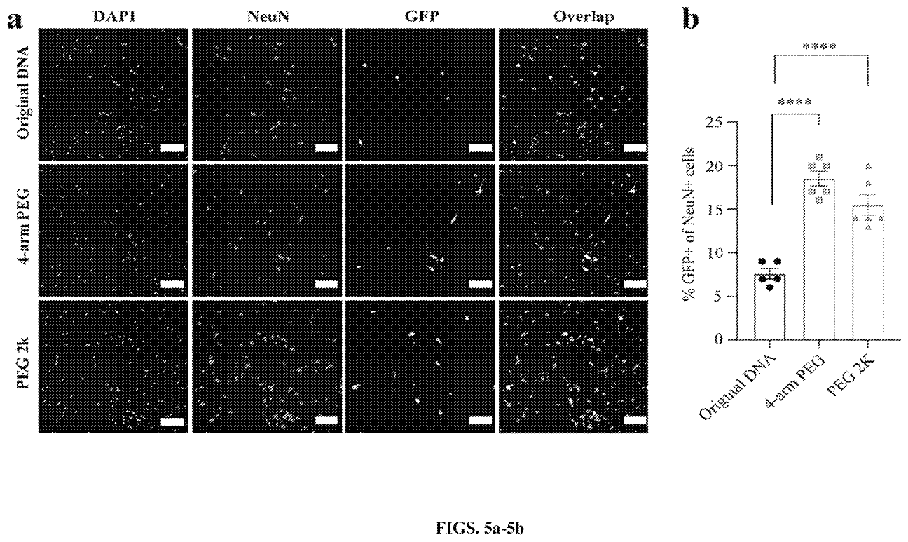

The gene transfection efficiency of b-DNA is enhanced in post-mitotic primary neuronal cells. Previous research has shown that non-viral vectors face significant obstacles in their ability to transfect post-mitotic cells, primarily due to the absence of nuclear membrane breakdown that occurs only during mitosis. The effective delivery of non-viral vectors to the nucleus is crucial for post-mitotic cells, as they need to traverse the nuclear envelope through nuclear pores and gain entry into the nucleus before transcription initiation can occur. The smaller size of b-DNA makes it more likely to pass through nucleopores compared to original DNA after escaping from the endosome into the cytoplasm, enhancing transfection efficiency. Here, this study evaluated the gene transfection efficiency of the RECAST strategy in embryonic mouse primary neurons. Commercial cationic polymers of jetOPTIMUS® were used to condense the b-DNA via electrostatic interaction. As shown in FIG. 5a, the b-DNA complexes exhibited enhanced gene transfection efficiency compared to polymers/original-DNA complexes. More specifically, the 4-arm PEG and PEG2k b-DNA/polymers complexes showed approximately a 2-fold increase in the percentage of neurons successfully expressing GFP (FIG. 5b). These results determined that our RECAST approach enables more efficient nuclear uptake and improved gene transfection efficiency in post-mitotic cells.

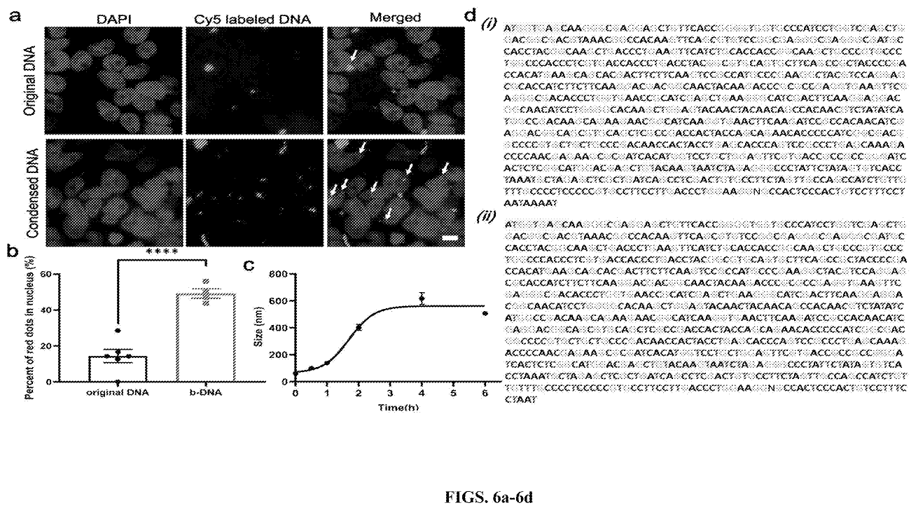

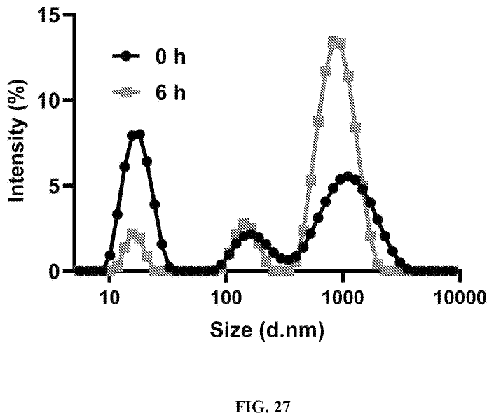

The nuclear import of b-DNA. To verify the diffusion and release of b-DNA in the nucleus, this study used confocal microscopy to image PEI delivered Cy5 labeled DNA at N/P of 10 for tracking their nucleus import. As shown in FIG. 6a, the b-DNA exhibited a higher red fluorescence signal in the nucleus than the original DNA after incubation for 6 h. The quantitative analysis showed that 49% of b-DNA was efficiently delivered to the nucleus, but only 15% of original DNA was successfully transported into the nucleus once they entered cells, as shown in FIG. 6b. These data determined that the b-DNA can more efficiently diffuse to and into the nucleus compared to the original DNA, due to the reduction in the DNA hydration radius after covalent crosslinking. Next, once the b-DNA diffuses into the nucleus, the traceless release of DNA is required for gene transcription to occur. The intracellular concentration of GSH can reach 10 mM compared to the extracellular concentration of ˜20 μM. Rapid hydrolysis rate of disulfide bond (hydrolysis half time is ˜30 mins) can occur under 10 mM GSH solution, which provided a good mechanism for the nucleus release. The recovery of DNA release was evaluated under the nucleus-mimic reductive environment (10 mM GSH). As shown in FIG. 6c, the size of b-DNA increased with the incubation time and recovered to a similar size with original DNA after incubation for 4 h (FIG. 27). This demonstrated that the disulfide in crosslinkers was gradually cleaved under the reduction of GSH and DNA can be completely released after incubation for 4 h. In addition, the released DNA was also collected for gene sequence analysis, as shown in FIG. 6d, where there is no change in the released DNA sequence compared to the original DNA sequence. This study shows that the DNA can be tracelessly released under intracellular GSH conditions to produce the correct gene transcripts without having any residues of crosslinkers or damage to the DNA structure.

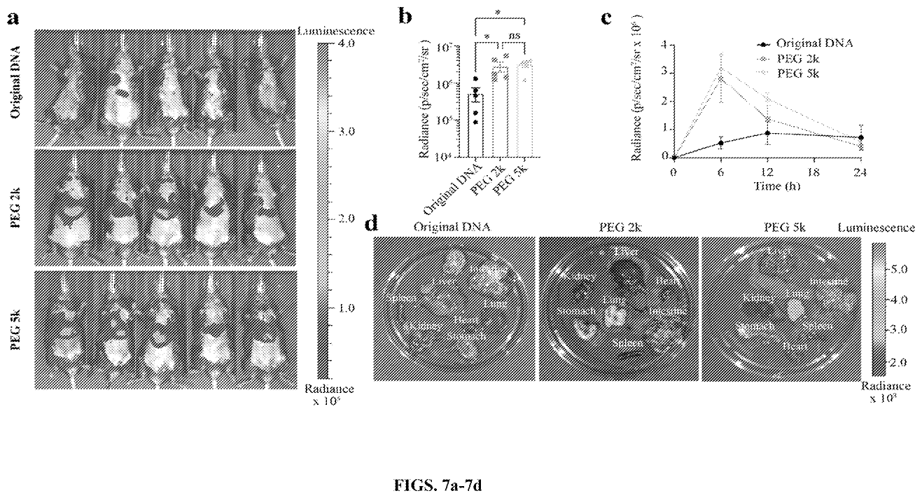

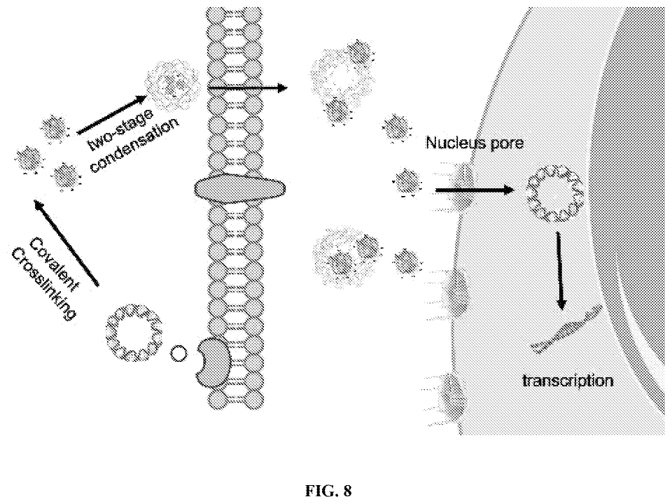

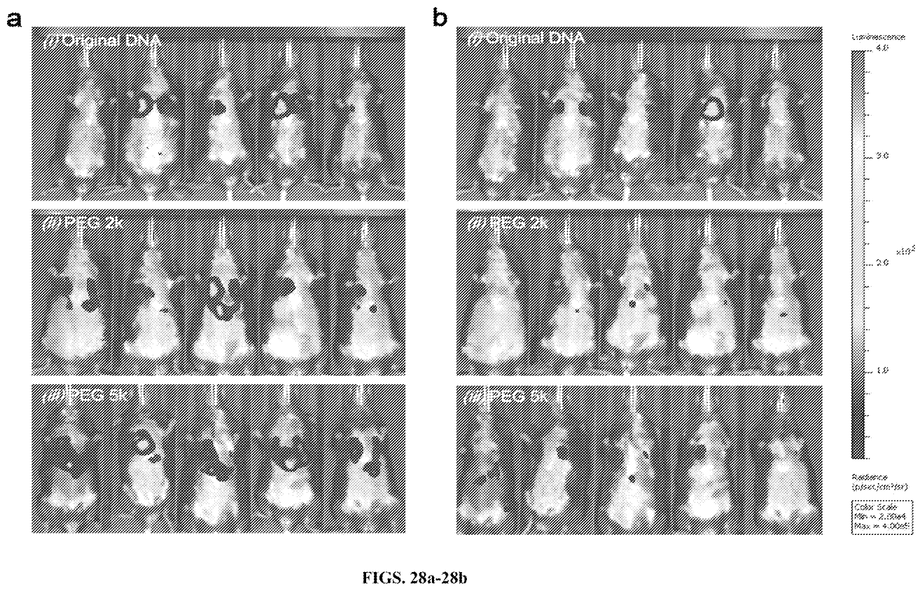

Improved lung-selective gene transfection by RECAST. The technology of delivering selective organ-targeted RNA via intravenous administration is revolutionizing nucleic acid therapeutics in a diverse range of diseases. Nevertheless, the application of organ-targeted DNA delivery has been limited by inefficient gene transfection due to the low nucleus transport efficiency of DNA. Therefore, the in vivo gene delivery efficiency of RECAST at targeted organs was also investigated after intravenous administration. PEG 2k and PEG 5K condensed b-DNA encoding for firefly luciferase were complexed with the commercial cationic polymer, in vivo-JetPEI, which is known to enrich DNA delivery in lungs. After a 6-hour period following tail vein injection of beta-DNA complexes, enhanced luciferase protein expression was observed in mice treated with RECAST compared to the original DNA treated group, with the luciferase signal increasing approximately four-fold. (FIGS. 7a, 7b). After that, the luciferase signal decreased with the treatment time (FIG. 7c and FIG. 28) due to the clearance by the immune system. The enhanced gene transfection efficiency can be attributed to improved nucleus delivery efficiency at target sites. Specifically, upon arrival in the targeted organs, the smaller size of the released b-DNA facilitated rapid and efficient traversal through nucleopores into the nucleus. This resulted in a more effective initiation of the transcription process compared to the original DNA. Additionally, the improved organ-selective gene delivery efficiency of RECAST was investigated by sacrificing mice 24 hours after treatment to extract organs for imaging. FIG. 7d shows that remarkable luciferase protein expression was observed only in the lung, indicating the utilization for targeted DNA therapeutics in clinical applications. The results of this study demonstrate that this RECAST approach is efficacious in achieving in vivo gene delivery.

New DNA crosslinker is designed and synthesized to condense the DNA into smaller size, and two stage condensation strategy is applied to achieve improved gene transfection efficiency. The crosslinkers can be used for gene function, and the two-stage condensation strategy can be used for gene/proteins/biomacromelucles delivery.

Gene therapy is promising for disease treatment, including cancer and inherited diseases. Viral vector-based gene therapy has been approved via FDA in recent years. However, limited via the low gene transfection efficiency, little non-viral vectors have been approved except the liposome-based mRNA vaccines for COVID treatment. This work innovates new DNA crosslinkers and two stage delivery strategy to achieve high gene transfection efficiency, thus, to decrease the gene therapy cost, increase the biosafety.

In conclusion, this study developed a RECAST to achieve enhanced DNA nucleus import and improved gene delivery efficiency. The DNA plasmids were firstly bundled to smaller-sized b-DNA via reversible covalent crosslinking and then packaged into the cationic polymers or liposomes via electronic interaction. Once the b-DNA was delivered and released in the cytoplasm, it became more easily diffused toward the nucleus in the cytoplasm and finally transported in the nucleus via nuclear pore complex channels. Meanwhile, the traceless release of DNA in the reductive nucleus environment enabled the correct DNA transcription to be initiated, thus achieving enhanced gene transfection efficiency without affecting the accuracy of the transcription. The in vitro tests determined that RECAST was an effective strategy to achieve improved gene transfection efficiency, including in primary neurons and with larger size plasmids. In addition, the enhanced organ-selective gene transfection of RECAST demonstrated the substantial value of non-viral gene delivery for clinical therapeutic applications. However, it is worth noting that the efficiency of DNA condensation is limited by the low reactivity of plasmids, thus constraining the minimum compressible size of DNA.

1. A nanocomposite comprising
a) a cationic vector; and
b) a chemically modified nucleic acid that comprises at least one nucleobase that is linked to a chemical crosslinker via a reversible covalent bond.

2. The nanocomposite of claim 1, wherein the reversible covalent bond is a carbamate bond.

3. The nanocomposite of claim 1, wherein the reversible covalent bond links the chemical crosslinker to an amine of the at least one nucleobase.

4. The nanocomposite of claim 1, wherein the chemical crosslinker is a p-nitrophenylcarbonate crosslinker.

5. The nanocomposite of claim 4, wherein the chemical crosslinker is disulfides (ethane-2,1-diyl)bis(4-nitrophenyl) bis(carbonate) (NPC).

6. The nanocomposite of claim 1, wherein the chemical crosslinker is PEGylated.

7. The nanocomposite of claim 6, wherein the chemical crosslinker is a PEGylated NPC crosslinker.

8. (canceled)

9. (canceled)

10. The nanocomposite of claim 1, wherein the chemically modified nucleic acid forms a bundle that is less than about 150 nm in size.

11. The nanocomposite of claim 1, wherein the cationic vector is a cationic polymer or a cationic liposome.

12. The nanocomposite of claim 11, wherein the cationic polymer is a polyethylene imine (PEI) polymer.

13. The nanocomposite of claim 12, wherein the PEI polymer is a branched polyethylenimine cationic polymer.

14. (canceled)

15. The nanocomposite of claim 1, wherein the nucleic acid is at least 5000 bp in length.

16. (canceled)

17. A pharmaceutical composition comprising the nanocomposite of claim 1.

18. A method of producing a nanocomposite for delivering a nucleic acid, said method comprising:
providing a chemical crosslinker;
contacting the nucleic acid with the chemical crosslinker thereby creating a chemically modified nucleic acid that comprises at least one nucleobase that is linked to a chemical crosslinker via a reversible covalent bond; and
contacting the chemically modified nucleic acid with a cationic vector thereby packaging the chemically modified nucleic acid into the cationic vector to produce the nanocomposite.

19-27. (canceled)

28. The method of claim 18, wherein the cationic vector is a cationic polymer or a cationic liposome.

29. (canceled)

30. The nanocomposite of claim 29, wherein the PEI polymer is a branched polyethylenimine cationic polymer.

31. The method of claim 30, wherein the cationic polymer is a polyethylenimine cationic polymer.

32. (canceled)

33. (canceled)

34. The method of claim 3, wherein the cationic polymer and the chemically modified nucleic acid is in contact at a nitrogen/phosphorus (N/P) ratio of about 5:1, 10:1, or 15:1.

35. The method of claim 30, wherein the cationic liposome and the chemically modified nucleic acid is in contact at a weight ratio of about 1:1, 1:2, or 1:3.

36. The method of claim 30, wherein the nucleic acid and the chemical crosslinker is in contact at a ratio in weight of about 1:4.

37-45. (canceled)
